(19) United States
(12) Patent Application Publication
(10) Pub. No.: US 2014/0154270 A1
(43) Pub. Date: Jun. 5, 2014

(54) PURIFICATION OF NON-HUMAN ANTIBODIES USING KOSMOTROPIC SALT ENHANCED PROTEIN A AFFINITY CHROMATOGRAPHY

(71) Applicants: Chen Wang, Shrewsbury, MA (US); Susan E. Lacy, Westborough, MA (US); Randolph Huelsman, North Chicago, IL (US)

(72) Inventors: Chen Wang, Shrewsbury, MA (US); Susan E. Lacy, Westborough, MA (US); Randolph Huelsman, North Chicago, IL (US)

(21) Appl. No.: 14/085,503

(22) Filed: Nov. 20, 2013

Related U.S. Application Data
(63) Continuation-in-part of application No. 13/898,984, filed on May 21, 2013.
(60) Provisional application No. 61/768,714, filed on Feb. 25, 2013.
(60) Provisional application No. 61/649,687, filed on May 21, 2012.

(51) Int. Cl. C07K 1/22

(52) U.S. Cl. CPC C07K 1/22
USPC 424/177.1; 530/390.5; 530/387.3

(57) ABSTRACT

The present invention is directed to methods for purifying a non-human antibody, or antigen binding portion thereof, exhibiting weak binding strength and low binding capacity for Protein A chromatography media. In one aspect, a kosmotropic salt solution is employed to promote the hydrophobic interaction between the non-human antibody, or antigen binding portion thereof, and the Protein A ligand, thereby enhancing the binding of the non-human antibody, or antigen binding portion thereof, to the Protein A chromatography media. In another aspect, the concentration of the non-human antibody, or antigen binding portion thereof, in a sample comprising the antibody, or antigen binding portion thereof, exposed to a Protein A chromatography media is increased to enhance the binding of the non-human antibody, or antigen binding portion thereof, on the Protein A chromatography media.

### RELATED APPLICATIONS

The present application is a continuation in part of U.S. application Ser. No. 13/898,984, filed May 21, 2013, and claims priority to U.S. Provisional Application No. 61/768,714, filed Feb. 25, 2013, and U.S. Provisional Application No. 61/649,687, filed on May 21, 2012, the disclosures of each of which are incorporated herein by reference in their entirety.

### BACKGROUND OF THE INVENTION

Protein A chromatographic resins are used in commercial purification processes for pharmaceutical grade monoclonal antibodies. The Protein A ligand is a cell wall protein derived from Staphylococcus aureus that comprises five homologous Ig binding domains (E, D, A, B and C) each of which are independently capable of binding to the Fc region of IgG1, IgG2 and IgG4. Each of the homologous IgG binding domains also have a high affinity for the Fab regions of some antibodies (Jansson et al., FEMS Immunol Med. Micro. (1998) 69-78). The Protein A ligand binds to mammalian antibodies, primarily through hydrophobic interactions along with hydrogen bonding and two salt bridges with the antibodies' Fc regions. The Protein A ligand is linked either directly, or indirectly, to a variety of matrices including cross-linked agarose, polyacrylamide in ceramic macrobeads, porous glass, polystyrenedivenylbenzene, polymeric and polymethacrylate (Hober et al., J Chromatography (2007) 848: 40-47). Thus, in the context of chromatographic purification, Protein A resins allow for the affinity-based retention of antibodies on a chromatographic support, while the majority of the components in a clarified harvest flow past the support and can be discarded. The retained antibodies can then be eluted from the chromatographic support by disrupting the antibody-Protein A interaction and subjected to further purification steps, e.g., those relying on charge (ion exchange chromatography), hydrophobic characteristics (hydrophobic interaction chromatography), and/or size (ultrafiltration).

Protein A-based affinity purification finds particular use in connection with a variety of commercially relevant immunoglobulin isotypes, particularly IgG1, IgG2, and IgG4. However, not all antibodies, including not all IgG1, IgG2, and IgG4 isotype immunoglobulins, are capable of binding Protein A with equal affinity. For instance, mouse IgG1 and canine, horse or cow IgG do not bind as strongly as a typical human IgG1 to Protein A. Consequently, those antibodies exhibiting weak binding strength for Protein A resin can result in low binding capacity under standard Protein A operating conditions, and, thus, demand a substantially larger Protein A column to process a given batch of antibody feed. Since Protein A capture is one of the most expensive steps in antibody downstream processing, using excess amount of Protein A resin will significantly increase its operating cost and create inefficiencies in conventional Protein A-based purification strategies. Hence, there is a present need for high-efficiency methods of purifying antibodies exhibiting weak binding strength and low binding capacity for Protein A resin.

### SUMMARY OF THE INVENTION

In one aspect, the present invention is directed to a method for producing a preparation including a non-human antibody, or antigen binding portion thereof, having a reduced level of at least one impurity, said method comprising (a) subjecting a sample comprising the non-human antibody, or antigen binding portion thereof, and at least one impurity to a first kosmotropic salt solution; (b) contacting the sample subjected to the kosmotropic salt solution to a Protein A affinity chromatography (PA) media; and (c) obtaining an elution fraction from the Protein A media; wherein the elution fraction comprises the non-human antibody, or antigen binding portion thereof, and has a reduced level of the at least one impurity.

In various embodiments, the non-human antibody, or antigen binding portion thereof, is a murine, canine, feline, bovine or equine antibody, or antigen binding portion thereof. In one particular embodiment, the non-human antibody, or antigen binding portion thereof, is a murine antibody, or antigen binding portion thereof. In another particular embodiment, the non-human antibody, or antigen binding portion thereof, is a canine antibody, or antigen binding portion thereof. In a further embodiment, the non-human antibody, or antigen binding portion thereof, is an IgG antibody, or antigen binding portion thereof. For example, the antibody, or antigen binding portion thereof, may be an IgG1, IgG2, IgG3 or IgG4 antibody. In a particular embodiment, the IgG antibody, or antigen binding portion thereof, is an IgG1 antibody, or antigen binding portion thereof.

In one embodiment, the non-human antibody, or antigen binding portion thereof, has a static binding capacity less than about 5 g, about 10 g, about 15 g, about 20 g, or about 25 g of antibody, or antigen binding portion thereof, per one liter of Protein A media. In another embodiment, the static binding capacity of the non-human antibody, or antigen binding portion thereof, increases by at least about 10%, about 25%, about 50%, about 75%, about 100%, about 150%, about 200%, about 300%, or about 400% when the sample is subjected to a kosmotropic solution.

In another embodiment, the non-human antibody, or antigen binding portion thereof, has a dynamic binding capacity less than about 5 g, about 10 g, about 15 g, about 20 g, or about 25 g of antibody, or antigen binding portion thereof, per one liter of Protein A media. In a further embodiment, the dynamic binding capacity of the non-human antibody, or antigen binding portion thereof, increases by at least about 10%, about 25%, about 50%, about 75%, about 100%, about 150%, about 200%, about 300%, or about 400% when the sample is subjected to a kosmotropic solution.

In various embodiments, the binding constant (K) of the non-human antibody, or antigen binding portion thereof, is at least 2, 3, 4, 5, 6, 7, 8, 9 or 10 fold lower than the binding constant (K) for a human antibody, for example a non-IgG3 IgG human antibody. In certain embodiments, the binding constant (K) of the non-human antibody, or antigen binding portion thereof, increases by at least about 10%, about 25%, about 50%, about 75%, about 100%, about 150%, about 200%, about 300%, or about 400% when the sample is subjected to a kosmotropic solution.

In a particular embodiment, the first kosmotropic salt includes a sulfate salt, a citrate salt, a phosphate salt, or a combination thereof. For example, the first kosmotropic salt solution can include a salt selected from the group consisting of ammonium sulfate, sodium sulfate, sodium citrate, potassium sulfate, potassium phosphate, sodium phosphate or a combination thereof.

In a particular embodiment, the sample is contacted to the Protein A chromatography media in the presence of a load buffer. In another embodiment, the Protein A chromatography media is exposed to an equilibration buffer and/or a wash buffer. In yet another embodiment, the elution fraction is obtained by contacting the Protein A chromatography media to an elution buffer. Alternatively or in combination, at least one of the load buffer, equilibration buffer and/or wash buffer include a second kosmotropic salt solution. In another embodiment, each of the load buffer, equilibration buffer and wash buffer include the second kosmotropic salt solution.

In a further embodiment, the load buffer, equilibration buffer and wash buffer comprise the same or substantially the same second kosmotropic salt solution. For example, the second kosmotropic salt can include a sulfate salt, a citrate salt, a phosphate salt, or a combination thereof. In a further example, the second kosmotropic salt solution includes a salt selected from the group consisting of ammonium sulfate, sodium sulfate, sodium citrate, potassium sulfate, potassium phosphate, sodium phosphate or a combination thereof.

In a particular embodiment, the first and second kosmotropic salt solutions are the same or substantially the same. For example, the first and/or second kosmotropic salt solution can include ammonium sulfate. In a further example, the first and/or second kosmotropic salt solution can include sodium sulfate. Alternatively, the first and/or second kosmotropic salt solution can include sodium citrate.

In a particular embodiment, the first and/or second kosmotropic salt solution has a concentration of between about 100 mM and 1500 mM. In another embodiment, the equilibration buffer, load buffer and/or the wash buffer have a pH between about 4.0 and 8.5 or between about 5.0 and 7.0. In another embodiment, the equilibration buffer, load buffer and the wash buffer are the same. In yet another embodiment, the equilibration buffer, load buffer and the wash buffer are substantially the same. For example, the salt concentration and/or the pH of the equilibration buffer, load buffer and/or wash buffer are within about 50%, 40%, 30%, 20%, 15%, 10% or 5% of the salt concentration and/or pH of each other.

In a further embodiment, the sample has a protein concentration greater than about 1 g/L, about 2 g/L, about 3 g/L, about 4 g/L, about 5 g/L, about 6 g/L, about 7 g/L, about 8 g/L, about 9 g/L or about 10 g/L.

In a particular embodiment, the elution fraction is substantially free of the at least one impurity. In one embodiment, the at least one impurity is a host cell protein. In another embodiment, the impurity is a process-related impurity. For example, the process-related impurity is selected from the group consisting of a host cell protein, a host cell nucleic acid, a media component, and a chromatographic material.

In one embodiment, the non-human antibody, or antigen binding portion thereof, is a humanized antibody or antigen-binding portion thereof, a chimeric antibody or antigen-binding portion thereof, or a multivalent antibody. In another embodiment, the non-human antibody, or antigen-binding fragment thereof, comprises a heavy chain constant region selected from the group consisting of IgG1, IgG2, IgG3, IgG4, IgM, IgA and IgE constant regions. In another embodiment, the non-human antibody, or antigen-binding fragment thereof, is selected from the group consisting of a Fab fragment, a F(ab′)2 fragment, a single chain Fv fragment, an SMIP, an affibody, an avimer, a nanobody, and a single domain antibody.

In one embodiment, the methods of the invention further include repeating steps (a)-(c) at least 1, 2, 3, 4, 5, 6, 7, 8, 9, 10, 11, 12, 13, 14, 15 or 20 times using the elution fraction having a reduced level of the at least one impurity.

In another embodiment of the present invention, wherein upon contacting the sample subjected to the kosmotropic salt solution to a Protein A media, a substantial portion of the non-human antibody, or antigen binding portion thereof, binds to the Protein A media. For example, the substantial portion of the non-human antibody, or antigen binding portion thereof, is at least about 50%, at least about 60%, at least about 70%, at least about 80%, at least about 90%, or at least about 100% of the antibody, or antigen binding portion thereof, in the sample.

In another embodiment, upon obtaining an elution fraction from the Protein A media, a substantial portion of the non-human antibody, or antigen binding portion thereof, is released from the Protein A media. For example, the substantial portion of the non-human antibody, or antigen binding portion thereof, released from the Protein A media is at least about 50%, at least about 60%, at least about 70%, at least about 80%, at least about 90% or about 100% of the amount of antibody, or antigen binding portion thereof, bound to the Protein A media.

In yet another embodiment, the yield of the non-human antibody, or antigen binding portion thereof, in the elution fraction is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

In a further embodiment of the present invention, upon contacting the sample subjected to the kosmotropic salt solution to a Protein A media, a substantial portion of the at least one impurity flows through the Protein A media. For example, the substantial portion of the at least one impurity that flows through the Protein A media is at least about 50%, at least about 55%, at least about 60%, at least about 65%, at least about 70%, at least about 75%, at least about 80%, at least about 85%, at least about 90%, at least about 95% or about 100% of the at least one impurity in the sample.

In one embodiment, the Protein A media is selected from the group consisting of MabSelect SuRe™, MabSelect, MabSelect SuRe LX, MabSelect Xtra, rProtein A Sepharose Fast Flow, Poros® MabCapture A, Amsphere™ Protein A JWT203, ProSep HC, ProSep Ultra, and ProSep Ultra Plus.

In a particular embodiment, the Protein A media comprises a column.

In a certain embodiment, about 10 g to about 100 g of the sample is contacted per one liter of Protein A media. In another embodiment, about 10 g to about 100 g of the non-human antibody, or antigen binding portion thereof, is contacted per one liter of HIC media.

In a particular embodiment, the concentration of the at least one impurity in the sample is about 100 ng to about 300 ng/mg antibody. In another embodiment, the level of the at least one impurity is reduced by at least 80%, at least 90%, at least 95%, at least 98%, at least 99%, or at least 99.9% of the at least one impurity in the sample. In yet another embodiment, the at least one impurity is reduced by at least 0.25, at least 0.5, at least 0.75, at least 1.0, at least 1.25, at least 1.5, at least 2.0, at least 2.5, at least 3.0 or at least 3.5 log reduction fraction.

In a particular embodiment, a precursor sample including the non-human antibody, or antigen binding portion thereof, has been subjected to hydrophobic interaction chromatography to generate the sample. Alternatively or in combination, the preparation including a non-human antibody, or antigen binding portion thereof, and having a reduced level of one impurity is subjected to hydrophobic interaction chromatography. In such embodiments, hydrophobic interaction chromatography may be performed using hydrophobic interaction media selected from the group consisting of CaptoPhenyl, Phenyl Sepharose™ 6 Fast Flow with low or high substitution, Phenyl Sepharose™ High Performance, Octyl Sepharose™ High Performance, Fractogel™ EMD Propyl, Fractogel™ EMD Phenyl, Macro-Prep™ Methyl, Macro-Prep™ t-Butyl, WP HI-Propyl (C3)™, Toyopearl™ ether, Toyopearl™ phenyl, Toyopearl™ butyl, ToyoScreen PPG, ToyoScreen Phenyl, ToyoScreen Butyl, ToyoScreen Hexyl, HiScreen Butyl FF, HiScreen Octyl FF, and Tosoh Hexyl.

In a particular embodiment, a precursor sample including the non-human antibody, or antigen binding portion thereof, has been subjected to ion exchange chromatography to generate the sample. Alternatively or in combination the preparation including a non-human antibody, or antigen binding portion thereof, and having a reduced level of one impurity is subjected to ion exchange chromatography. In such embodiments, ion exchange chromatography may be performed using ion exchange chromatography media selected from the group consisting of (i) a cation exchange media, for example, comprising carboxymethyl (CM), sulfoethyl (SE), sulfopropyl (SP), phosphate (P) or sulfonate (S) ligands, and (ii) an anion exchange media, for example, comprising diethylaminoethyl (DEAE), quaternary aminoethyl (QAE) or quaternary amine (Q) group ligands.

In one embodiment, a precursor sample including the non-human antibody, or antigen binding portion thereof, has been subjected to mixed mode chromatography to generate the sample. Alternatively or in combination, the method involves subjecting the preparation including the non-human antibody, or antigen binding portion thereof, and having a reduced level of one impurity to mixed mode chromatography, for example, using CaptoAdhere resin.

In one embodiment, a precursor sample including the non-human antibody, or antigen binding portion thereof, has been subjected to a filtration step to generate the sample. Alternatively or in combination, the method involves subjecting the preparation including the non-human antibody, or antigen binding portion thereof, and having a reduced level of one impurity to a filtration step, for example, a depth filtration step, a nanofiltration step, an ultrafiltration step, and an absolute filtration step, or a combination thereof.

In one aspect, the present invention is directed to a pharmaceutical composition including the preparation produced by any of the foregoing methods, and a pharmaceutically acceptable excipient. In another aspect, the present invention is directed to a pharmaceutical composition including a non-human antibody, or antigen binding portion thereof, and a reduced level of at least one impurity, for example, host cell protein. In a particular aspect, the present invention is directed to a pharmaceutical composition including a canine antibody, or antigen-binding portion thereof, and a reduced level of host cell protein.

In a particular aspect, the present invention is directed to a pharmaceutical composition comprising a non-human antibody, or antigen binding portion thereof, and a reduced level of at least one impurity. For example, the non-human antibody, or antigen binding portion thereof, is selected from the group consisting of a murine, canine, feline, bovine or equine antibody, or antigen binding portion thereof. Alternatively, or in combination, the non-human antibody, or antigen binding portion thereof, is an IgG antibody, or antigen binding portion thereof. In a particular embodiment, the IgG antibody, or antigen binding portion thereof, is an IgG1 antibody, or antigen binding portion thereof. In another embodiment, the impurity is a host cell protein. In another embodiment, the composition comprises less than about 10%, 9%, 8%, 7%, 6%, 5%, 4%, 3%, 2%, 1.5%, 1.4%, 1.3%, 1.2%, 1.1%, 1%, 0.5%, or less total impurities, e.g., host cell proteins.

In another aspect, the invention comprises a canine IgG antibody, or antigen binding portion thereof, having less than about 10%, 9%, 8%, 7%, 6%, 5%, 4%, 3%, 2%, 1.5%, 1.4%, 1.3%, 1.2%, 1.1%, 1%, 0.5%, of host cell protein.

### BRIEF DESCRIPTIONS OF THE DRAWINGS

FIG. 1 depicts a two-column purification process for the present invention.

FIG. 2 depicts a three-column purification process for the present invention.

FIG. 3 depicts the effects of the load protein concentration on the static binding capacity of a weak Protein A binding monoclonal antibody (i.e., canine Mab A) to MabSelect SuRe Protein A resin.

FIG. 4 depicts the effect of various kosmotropic salts and their concentrations on static binding capacity of a weak Protein A binding monoclonal antibody (i.e., canine Mab A) to MabSelect SuRe Protein A resin.

FIG. 5 depicts the effects of (NH₄)₂SO₄, protein concentration and flow rates on dynamic binding capacity of a weak Protein A binding monoclonal antibody (i.e., canine Mab A) on MabSelect SuRe Protein A column.

FIG. 6 depicts the effect of various kosmotropic salt solution comprising ammonium sulfate, sodium sulfate, or sodium citrate on the binding capacity of a weak Protein A binding monoclonal antibody (i.e., canine Mab A) on MabSelect SuRe Protein A column

FIG. 7 depicts the effect of a kosmotropic salt solution comprising various concentrations of ammonium sulfate on the dynamic binding capacity of a weak Protein A binding monoclonal antibody (i.e., canine Mab A) on MabSelect SuRe Protein A column with load titer 4.7-5.8 g/L.

FIG. 8 depicts the effect of a kosmotropic salt solution comprising various concentrations of ammonium sulfate on HCP levels in the MabSelect SuRe Protein A eluate for a weak Protein A binding monoclonal antibody (i.e., canine Mab A). Load titer 4.7-5.8 g/L containing ˜200,000 ng/mg HCP.

FIG. 9 depicts the dynamic binding capacity (DBC) of a weak Protein A binding monoclonal antibody (i.e., canine MAb A) on ProSep Ultra Plus Protein A resin in the absence and presence of kosmotropic salt.

### DETAILED DESCRIPTION OF THE INVENTION

The present invention is directed to methods for purifying a non-human antibody, or antigen binding fragment thereof, from a sample. In particular, the present invention relates to methods for purifying an antibody, or antigen binding portion thereof, exhibiting weak binding strength and low binding capacity for Protein A chromatography media. In certain embodiments, the present invention is directed to enhancing the amount of a non-human antibody, or antigen binding portion thereof, retained on a Protein A chromatography media, where such antibody exhibits weak binding strength and low binding capacity for such media.

In part, the present invention is predicated upon the finding that by exposing a sample including a non-human antibody, or an antigen binding fragment thereof, that exhibits weak binding strength and/or low binding capacity for Protein A chromatography media to a kosmotropic salt, the antibody, or antigen binding portion thereof, exhibits improved binding to the Protein A chromatography media and, thereby, allows for improved purification thereof. Accordingly, in one aspect, a kosmotropic salt solution is employed to promote the hydrophobic interaction between the non-human antibody, or antigen binding portion thereof, and the Protein A ligand, thereby enhancing the binding of the antibody to the Protein A chromatography media.

The present invention is further predicated, at least in part, on the finding that by increasing the concentration of the non-human antibody, or antigen binding portion thereof, in a sample, the level of antibody, or antigen binding portion thereof, bound to the Protein A chromatography media increases, thereby allowing for improved purification thereof. Accordingly, in one aspect, the concentration of the non-human antibody, or antigen binding portion thereof, in a sample is increased to enhance the binding of the antibody to the Protein A chromatography media.

In certain embodiments, a combination of a kosmotropic salt solution and an increased concentration of the non-human antibody is employed to enhance the retention of the antibody on the Protein A chromatography media and substantially improve purification of the antibody, or antigen binding portion thereof.

In certain embodiments, the purification strategies of the present invention may include one or more additional chromatography and/or filtration steps to achieve a desired degree of purification. For example, in certain embodiments, the chromatography step(s) can include one or more steps of ion exchange chromatography and/or hydrophobic interaction chromatography.

### DEFINITIONS

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms, for example, those characterized by “a” or “an”, shall include pluralities, e.g., one or more impurities. In this application, the use of “or” means “and/or”, unless stated otherwise. Furthermore, the use of the term “including,” as well as other forms of the term, such as “includes” and “included”, is not limiting. Also, terms such as “element” or “component” encompass both elements and components comprising one unit and elements and components that comprise more than one unit unless specifically stated otherwise.

As used herein, the term “sample”, refers to a liquid composition including the non-human antibody and one or more impurities. In a particular embodiment, the sample is a “clarified harvest”, referring to a liquid material containing an antibody, for example, a non-human antibody such as a canine antibody, that has been extracted from cell culture, for example, a fermentation bioreactor, after undergoing centrifugation to remove large solid particles and subsequent filtration to remove finer solid particles and impurities from the material.

In various embodiments, the sample may be partially purified. For example, the sample may have already been subjected to any of a variety of art recognized purification techniques, such as chromatography, e.g., ion exchange chromatography, mixed mode chromatography, and/or hydrophobic interaction chromatography, or filtration, e.g., depth filtration, nanofiltration, ultrafiltration and/or absolute filtration.

In various embodiments, the sample may be subjected to any of a variety of art recognized techniques to increase the concentration of the antibody, for example, a non-human antibody such as a canine antibody. An example of techniques used to increase the concentration of the antibody include membrane ultrafiltration.

In various embodiments, the sample may be exposed to a kosmotropic salt solution prior to contacting the sample with the Protein A media.

The term “precursor sample”, as used herein refers to a liquid composition containing the non-human antibody and, optionally, one or more impurities, either derived from the clarified harvest, or a partially purified intermediate sample that is subject to a purification or treatment step prior to being subjected to Protein A affinity chromatography. Impurities in a precursor sample may be derived from the production, purification or treatment of the non-human antibody prior to subjecting the resulting sample to Protein A affinity chromatography.

The term “antibody”, as used herein refers to a target antibody present in a sample, purification of which is desired. In various embodiment, the antibody is an antibody or antigen-binding fragment thereof. In a particular embodiment, the antibody is a non-human antibody, such as a canine, feline, murine, equine or bovine antibody.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The term “antibody”, as used herein, also includes alternative antibody and antibody-like structures, such as, but not limited to, dual variable domain antibodies (DVD-Ig).

The term “antigen-binding portion” of an antibody (or “antibody portion”) includes fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment comprising the VL, VH, CL and CH1 domains; (ii) a F(ab′)2 fragment, a bivalent fragment comprising two Fab fragments linked by a disulfide bridge at the hinge region; (iii) a Fd fragment comprising the VH and CH1 domains; (iv) a Fv fragment comprising the VL and VH domains of a single arm of an antibody, (v) a dAb fragment (Ward et al., (1989) Nature 341:544-546, the entire teaching of which is incorporated herein by reference), which comprises a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883, the entire teachings of which are incorporated herein by reference). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123, the entire teachings of which are incorporated herein by reference). Still further, an antibody may be part of a larger immunoadhesion molecule, formed by covalent or non-covalent association of the antibody with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101, the entire teaching of which is incorporated herein by reference) and use of a cysteine residue, a marker peptide and a C-terminal polyhistidine tag to make bivalent and biotinylated scFv molecules (Kipriyanov, S. M., et al. (1994) Mol. Immunol. 31:1047-1058, the entire teaching of which is incorporated herein by reference). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. In one aspect, the antigen binding portions are complete domains or pairs of complete domains.

An “isolated antibody” includes an antibody that is substantially free of other antibodies having different antigenic specificities. Moreover, an isolated antibody may be substantially free of other cellular material and/or chemicals.

In various embodiments, the antibody, or antigen binding portion thereof, is a murine, feline, canine, bovine or equine antibody, or antigen binding portion thereof. In a particular embodiment, the antibody, or antigen binding portion thereof, is a murine antibody, or antigen binding portion thereof. In another embodiment, the antibody, or antigen binding portion thereof, is a canine antibody, or antigen binding portion thereof. In particular embodiments, the antibody, or antigen binding portion thereof is a murine, feline, canine, bovine or equine IgG antibody, e.g., an IgG1, IgG2, IgG3 or IgG4 antibody. In a particular embodiment, the antibody, or antigen binding portion thereof, is a murine, feline, canine, bovine or equine IgG1 antibody, or antigen binding portion thereof.

The term “impurity”, as used herein refers to any foreign or objectionable molecule, including a biological macromolecule such as a DNA, an RNA, or a protein other than the antibody being purified. Exemplary impurities include, for example, host cell proteins; proteins that are part of an absorbent used for chromatography; endotoxins; and viruses.

The methods of the invention serve to generate a preparation comprising an antibody and having a reduced level of impurity. As used herein a “reduced level of impurity” refers to a composition comprising reduced levels of an impurity as compared to the levels of the impurity in the sample prior to purification by the methods of the present invention. In another embodiment, the methods of the invention generate a preparation comprising an antibody and having a reduced level of total impurity. As used herein a “reduced level of total impurity” refers to a composition comprising reduced levels of total impurity as compared to the levels of the impurity in the sample prior to purification by the methods of the present invention. In one embodiment, a preparation having a reduced level of total impurity is free of impurities or substantially free of impurities.

The present invention is further directed to low impurity compositions and methods of generating the same, for example, low impurity compositions of a non-human antibody. The term “low impurity composition,” as used herein, refers to a composition comprising an antibody, wherein the composition contains less than about 15% total impurities. For example, a low impurity composition may contain about 10%, 9%, 8%, 7%, 6%, 5%, 4%, 3%, 2%, 1.5%, 1.4%, 1.3%, 1.2%, 1.1%, 1%, 0.5%, or less total impurities. In a particular embodiment, a low impurity composition comprises about 5%, 4%, 3%, 2.5%, 2.4%, 2.3%, 2.2%, 2.1%, 2%, 1.5%, 1.4%, 1.3%, 1.2%, 1.1%, 1%, 0.5%, 0.1%, or less total impurities.

The term “non-low impurity composition,” as used herein, refers to a composition comprising a non-human antibody, which contains more than about 15% total impurity. For example, a non-low impurity composition may contain about 15%, 16%, 17%, 18%, 19%, 20%, 21%, 22%, 23%, 24%, 25%, or more total impurities.

In one embodiment, a low impurity composition has improved biological and functional properties, including increased efficacy in the treatment or prevention of a disorder in a subject, for example, a non-human subject.

In a particular embodiment, the impurity is a process-related impurity. As used herein, the term “process-related impurity,” refers to impurities that are present in a composition comprising a non-human antibody but are not derived from the antibody itself. Process-related impurities include, but are not limited to, host cell proteins (HCPs), host cell nucleic acids, chromatographic materials, and media components. A “low process-related impurity composition,” as used herein, refers to a composition comprising reduced levels of process-related impurities as compared to a composition wherein the impurities were not reduced. For example, a low process-related impurity composition may contain about 10%, 9%, 8%, 7%, 6%, 5%, 4%, 3%, 2%, 1%, 0.5%, 0.4%, 0.3%, 0.2%, 0.1% or less process-related impurities. In one embodiment, a low process-related impurity composition is free of process-related impurities or is substantially free of process-related impurities.

In one embodiment, the impurity is a host cell protein. The term “host cell protein” (HCP), as used herein, is intended to refer to non-antibody proteinaceous impurities derived from host cells, for example, host cells used to produce the antibody.

In one embodiment, the impurity is a host cell nucleic acid. The term “host cell nucleic acids”, as used herein, is intended to refer to nucleic acids derived from host cells, for example, host cells used to produce the antibody.

The term “equilibration buffer”, as used herein refers to a salt solution passed through the Protein A media prior to contacting the sample with the Protein A media. In some embodiments, the equilibration buffer is used to establish a particular pH and/or salt concentration of the solution surrounding the Protein A media prior to addition of the load buffer and sample. In one embodiment, the equilibration buffer comprises a kosmotropic salt.

The term “load buffer”, as used herein refers to a salt solution passed through the Protein A media upon contacting the sample with the Protein A media. In certain embodiments, the load buffer is passed through the Protein A media simultaneously or substantially simultaneously with passage of the sample through the Protein A media. In certain embodiments, the load buffer is combined with the sample prior to passage through the Protein A media. In one embodiment, the load buffer comprises a kosmotropic salt.

The term “wash buffer”, as used herein refers to a salt solution passed through the Protein A media during the wash phase. In one embodiment, the wash buffer comprises a kosmotropic salt.

The term “wash fraction”, as used herein refers to the liquid eluted from the column upon washing the Protein A media with the wash buffer. The wash fraction may also include wash buffer that passes through the Protein A media during the wash phase and the substantial portion of the impurity that does not bind to the Protein A media.

The term “elution buffer”, as used herein refers to a salt solution passed through the Protein A media during the elution phase.

The term “elution fraction”, as used herein refers to the liquid eluted from the column, for example, upon contacting the Protein A media with the elution buffer. According to the methods of the present invention, the elution fraction includes the non-human antibody, or antigen binding portion thereof, that is released from the Protein A media and has a reduced level of at least one impurity.

The term “kosmotropic”, as used herein refers to a salt (e.g., ammonium sulfate, sodium sulfate, sodium citrate) which contributes to the stability and structure of water-water interactions and causes water molecules to favorably interact with macromolecules such as proteins. Intermolecular interactions are also stabilized by kosmotropic salts. In various embodiments, a kosmotropic salt is employed to enhance the hydrophobic interaction between the antibody and the Protein A affinity media.

The term “load challenge”, as used herein refers to the total mass of sample (e.g., non-human antibody and at least one impurity) loaded onto the column in chromatography applications or applied to the resin in batch binding, measured in units of mass of product per unit volume of resin.

The phrase “dynamic binding capacity”, as used herein, refers to the amount of non-human antibody that can bind to a chromatography media under flow conditions upon breakthrough of 5% of the total protein load. This value is always lower than the static or saturation capacity.

The phrase “static binding capacity” as used herein, refers to the amount of non-human antibody a column can bind if every available binding site is utilized. This is determined by loading a large excess of antibody either at very slow flow rates or after prolonged incubation in a closed system.

The phrase “weak binding strength” and “weak binding”, as used herein, is intended to refer to an antibody, for example, a non-human antibody, exhibiting a reduced binding capacity as compared to a typical human IgG antibody, except for human IgG3 antibodies, e.g., such weak binding strength leads to about 2-10 fold lower binding capacity than that expected for a typical human IgG antibody, except for human IgG3 antibodies, for a particular chromatographic resin, e.g., a Protein A resin, and which would lead to inefficient purification under conventional purification conditions. For example, in certain embodiments, the weak binding antibody is characterized by having a binding constant for a standard Protein A resin at least 5, 6, 7, 8, 9, or 10 fold lower than that for a typical human IgG antibody.

The phrase “low binding capacity”, as used herein, is intended to refer to an antibody, for example, non-human antibody, exhibiting a reduced static binding capacity and/or a reduced dynamic binding capacity for the Protein A media. For example, as compared to a typical human IgG antibody, except for human IgG3 antibodies, such weak binding strength leads to about 2-10 fold lower binding capacity than that expected for a typical human IgG antibody, except for human IgG3 antibodies, for a particular chromatographic resin, e.g., a Protein A resin, and which would lead to inefficient purification under conventional purification conditions. In one embodiment, the Protein A media binds less than about 5 g/L, about 10 g/L, about 15 g/L, about 20 g/L, or about 25 g/L of the antibody.

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

### Antibody Purification

### Antibody Purification Generally

The present invention provides a method for producing a preparation including a non-human antibody, and having a reduced level of at least one impurity, e.g., a host cell protein, by contacting a sample including the non-human antibody and at least one impurity, to a Protein A affinity chromatography media.

In certain embodiments, the compositions of the present invention include, but are not limited to, a preparation comprising a non-human antibody having a reduced level of at least one impurity. For example, but not by way of limitation, the present invention is directed to preparations of a non-human antibody (e.g., a canine antibody) having a reduced level of at least one impurity, for example, host cell protein. Such preparations having a reduced level of at least one impurity address the need for improved product characteristics, including, but not limited to, product stability, product safety and product efficacy. In further embodiments, compositions of the present invention include pharmaceutical compositions comprising the preparation produced by the methods of the invention (e.g., antibody having a reduced level of the at least on impurity) and a pharmaceutically acceptable carrier.

In certain embodiments, the purification process of the invention begins at the separation step when the non-human antibody has been produced using production methods described herein and/or by alternative production methods conventional in the art. Once a clarified solution or sample including the non-human antibody has been obtained, separation of the non-human antibody from at least one impurity, such as process-related impurities, e.g., other proteins produced by the cell, can be performed using a Protein A affinity separation step, or a combination of a Protein A affinity separation step and one or more purification techniques, including filtration and/or affinity, ion exchange, hydrophobic interaction chromatography and/or mixed mode chromatographic step(s), as outlined herein. Table 1 summarizes one embodiment of a purification scheme.

TABLE 1
Purification steps

| Purification step | Purpose |
|---|---|
| Primary recovery | Clarification of cell culture sample matrix by |
| (Centrifugation and/ | removing cells and cell debris |
| or Depth filtration) | |
| Ultrafiltration | Concentrating antibody |
| Viral inactivation | Inactivation of encapsulated virus by detergent or |
| | low pH |
| Protein A Affinity | Antibody capture, host cell protein and associated |
| chromatography | impurity reduction |
| Depth filtration | Remove turbidity/precipitates and impurities |
| Ion exchange | Reduction of host cell proteins, DNA, aggregates, |
| chromatography | leached protein A and virus |
| (anion or cation) | |
| Hydrophobic | Reduction of antibody aggregates, host cell proteins, |
| interaction | DNA, leached protein A and virus |
| chromatography | |
| Viral filtration | Removal of virus, if present |
| Ultrafiltration/ | Concentrate and formulate antibody |
| Diafiltration | |

### Primary Recovery

In certain embodiments, the initial steps of the purification methods of the present invention involve the clarification and primary recovery of the non-human antibody, for example, a non-human antibody such as a canine antibody, following production. In certain embodiments, the primary recovery will include one or more centrifugation steps to separate the non-human antibody from cells and cell debris. Centrifugation of the non-human antibody containing composition can be run at, for example, but not by way of limitation, 7,000×g to approximately 12,750×g. In the context of large scale purification, such centrifugation can occur on-line with a flow rate set to achieve, for example, but not by way of limitation, a turbidity level of 150 NTU in the resulting supernatant. Such supernatant can then be collected for further purification, or in-line filtered through one or more depth filters for further clarification of the sample.

In certain embodiments, the primary recovery will include the use of one or more depth filtration steps to clarify the sample and thereby aid in purifying the non-human antibody in the present invention. In other embodiments, the primary recovery will include the use of one or more depth filtration steps post centrifugation to further clarify the sample. Non-limiting examples of depth filters that can be used in the context of the instant invention include the Millistak+ X0HC, F0HC, D0HC, A1HC, B1HC depth filters (EMD Millipore), Cuno™ model 30/60ZA, 60/90 ZA, VR05, VR07, delipid depth filters (3M Corp.). A 0.2 μm filter such as Sartorius's 0.45/0.2 μm Sartopore™ bi-layer or Millipore's Express SHR or SHC filter cartridges typically follows the depth filters.

In certain embodiments, the primary recovery process can also be a point at which to reduce or inactivate viruses that can be present in the sample. For example, any one or more of a variety of methods of viral reduction/inactivation can be used during the primary recovery phase of purification including heat inactivation (pasteurization), pH inactivation, solvent/detergent treatment, UV and γ-ray irradiation and the addition of certain chemical inactivating agents such as β-propiolactone or e.g., copper phenanthroline as in U.S. Pat. No. 4,534,972. In certain embodiments of the present invention, the sample is exposed to detergent viral inactivation during the primary recovery phase. In other embodiments, the sample may be exposed to low pH inactivation during the primary recovery phase.

In those embodiments where viral reduction/inactivation is employed, the sample can be adjusted, as needed, for further purification steps. For example, following low pH viral inactivation, the pH of the sample is typically adjusted to a more neutral pH, e.g., from about 4.5 to about 8.5, prior to continuing the purification process. Additionally, the mixture may be diluted with water for injection (WFI) to obtain a desired conductivity.

### Protein A Affinity Chromatography

The instant invention features methods for producing a preparation comprising an antibody (e.g., a non-human antibody, such as a canine antibody) having a reduced level of at least one impurity, for example, host cell proteins, from a sample comprising the antibody and at least one impurity by contacting the sample with Protein A media.

In one aspect, the present invention provides a method for producing a preparation including an antibody, e.g., a non-human antibody, such as a canine antibody, and having a reduced level of at least one impurity, e.g., a host cell protein, by (a) subjecting a sample comprising the antibody and at least one impurity to kosmotropic salt solution; (b) contacting the sample subjected to kosmotropic salt solution to a Protein A affinity chromatography (PA) media; and (c) obtaining an elution fraction from the Protein A media wherein the elution fraction comprises the antibody and has a reduced level of the at least one impurity.

According to the present invention, Protein A purification of an antibody, for example, a non-human antibody such as a canine antibody, comprises reversible binding of the non-human antibody in the presence of a kosmotropic salt while a substantial portion of the one or more impurities flow past the Protein A media and can be discarded. In the absence of the kosmotropic salts, the antibody exhibits weak binding strength and/or low binding capacity (e.g., static binding capacity and/or dynamic binding capacity) for the Protein A media resulting in inefficient purification of the antibody from the at least one impurity. The efficiency of the purification of the non-human antibody can be further improved by increasing the concentration of the sample, prior to contacting it with the Protein A media. Thus, Protein A affinity chromatography steps, such as those disclosed herein, can be used to remove a variety of impurities, for example, process-related impurities (e.g., DNA, host cell proteins) from a sample comprising an antibody.

In certain embodiments, it will be advantageous to determine the dynamic binding capacity (DBC) of the Protein A resin in order to tailor the purification to the particular antibody. For example, but not by way of limitation, the DBC of a MabSelect SuRe™ column can be determined either by a single flow rate load or dual-flow load strategy. The single flow rate load can be evaluated at a velocity of about 335 cm/hr throughout the entire loading period. The dual-flow rate load strategy can be determined by loading the column up to about 24 mg protein/mL resin at a linear velocity of about 335 cm/hr, then reducing the linear velocity to 220 cm/hr to allow longer residence time for the last portion of the load.

In one embodiment, in the absence of kosmotropic salts, the non-human antibody has a low static binding capacity for the Protein A media. For example, in various embodiments, the static binding capacity of the non-human antibody for the Protein A media is less than about 1 g/L, about 5 g/L, about 10 g/L, about 15 g/L, about 20 g/L, or about 25 g/L of Protein A media.

In another embodiment of the present invention, in the presence of the kosmotropic salts, the non-human antibody has an increased static binding capacity for the Protein A media. For example, in various embodiments, the static binding capacity will increase by at least about 10%, about 25%, about 50%, about 75%, about 100%, about 150%, about 200%, about 300%, or about 400%. As a result, the static binding capacity of the antibody for the Protein A media will be greater than about 5 g/L, 10 g/L, 15 g/L, 20 g/L, 25 g/L, about 30 g/L, about 35 g/L, about 40 g/L, about 45 g/L, about 50 g/L, about 55 g/L, about 60 g/L, about 65 g/L, about 70 g/L, about 80 g/L.

In another embodiment of the present invention, in the absence of kosmotropic salts, the non-human antibody has a low dynamic binding capacity for the Protein A media. For example, in various embodiments, the dynamic binding capacity of the non-human antibody for the Protein A media is less than about 1 g/L, about 5 g/L, about 10 g/L, about 15 g/L, about 20 g/L, or about 25 g/L of Protein A media.

In another embodiment of the present invention, in the presence of the kosmotropic salts, the non-human antibody has an increased dynamic binding capacity for the Protein A media. For example, in various embodiments, the dynamic binding capacity will increase by at least about 10%, about 25%, about 50%, about 75%, about 100%, about 150%, about 200%, about 300%, or about 400%. As are result, the dynamic binding capacity of the antibody for the Protein A media will be greater than about 5 g/L, 10 g/L, 15 g/L, 20 g/L, 25 g/L, about 30 g/L, about 35 g/L, about 40 g/L, about 45 g/L, about 50 g/L, about 55 g/L, about 60 g/L, about 65 g/L, about 70 g/L, about 80 g/L.

In another embodiment of the invention, in the absence of kosmotropic salts, the antibody has a weak binding strength for the Protein A media. For instance the antibody may bind to the Protein A media with a binding strength that is about 2, 3, 4, 5, 6, 7, 8, 9 or 10 fold lower than expected for a typical IgG antibody. In a particular embodiment, the binding constant (K) of the non-human antibody, or antigen binding portion thereof, increases by at least about 10%, about 25%, about 50%, about 75%, about 100%, about 150%, about 200%, about 300%, or about 400% when the sample is subjected to a kosmotropic solution.

In certain embodiments, the non-human antibody, or antigen binding portion thereof, is a murine, canine, feline, bovine or equine antibody, or antigen binding portion thereof. In another embodiment, the antibody, or antigen binding portion thereof, is a murine antibody, or antigen binding portion thereof. In yet another embodiment, the antibody, or antigen binding portion thereof, is a canine antibody, or antigen binding portion thereof. In another embodiment, the antibody, or antigen binding portion thereof, is an IgG antibody, for example, an IgG1, IgG2, IgG3 or IgG4 antibody, or antigen binding portion thereof. In a particular embodiment, the IgG antibody, or antigen binding portion thereof, is an IgG1 antibody, or antigen binding portion thereof.

In certain embodiments, an increased concentration of the antibody as compared to conventional purification strategies is loaded onto the Protein A media. For antibodies with relatively low static and or dynamic binding capacity for the Protein A media, such an increased load concentration of the antibody enhances its binding capacity to the Protein A media. In certain of such embodiments, the antibody in the sample matrix that is contacted to a Protein A media has a concentration of from about 1 g/L to about 10 g/L. In certain embodiments the concentration is from about 1.5 g/L to about 8 g/L, about 1.5 g/L to about 5.8 g/L, about 1.7 g/L to about 5.8 g/L, about 1.9 g/L to about 5.45 g/L, about 1.9 g/L to about 4.95 g/L, about 1.9 g/L to about 4.7 g/L, about 1.9 g/L to about 4.5 g/L, or about 1.9 g/L to about 3.6 g/L. In certain embodiments, the concentration is about 2 g/L, about 3 g/L, about 4 g/L, about 5 g/L, about 6 g/L, about 7 g/L, about 8 g/L, or about 9 g/L.

In certain embodiments, the sample comprising the antibody is exposed to a kosmotropic salt solution prior to contacting with a Protein A media. The kosmotropic salt solution comprises at least one kosmotropic salt. For example, the kosmotropic salt may be a sulfate salt, a citrate salt, a phosphate salt, or a combination thereof. In a particular embodiment, the kosmotropic salt solution includes a salt selected from the group consisting of ammonium sulfate, sodium sulfate, sodium citrate, potassium sulfate, potassium phosphate, sodium phosphate or a combination thereof. In one embodiment, the kosmotropic salt is ammonium sulfate. In another embodiment, the kosmotropic salt is sodium sulfate. In yet another embodiment, the kosmotropic salt is sodium citrate.

In various embodiments, the kosmotropic salt is present in the kosmotropic salt solution at a concentration of from about 100 mM to about 1500 mM. In one embodiment, the kosmotropic salt is present in the kosmotropic salt solution at a concentration of about 300 min.

In performing the Protein A separation, the sample may be contacted with the Protein A media, e.g., using a batch purification technique or using a column. For example, in the context of chromatographic separation, a chromatographic apparatus, commonly cylindrical in shape, is employed to contain the chromatographic media (e.g., Protein A media) prepared in an appropriate buffer solution.

There are several commercial sources for Protein A media. One suitable media is MabSelect SuRe™ from GE Healthcare. A non-limiting example of a suitable column packed with MabSelect SuRe™ is an about 1.0 cm diameter×about 22 cm long column (˜17 mL bed volume). This size column can be used for small scale purifications and can be compared with other columns used for scale ups. For example, a 20 cm×22 cm column whose bed volume is about 6.9 L can be used for larger purifications. Regardless of the column, the column can be packed using a suitable resin such as MabSelect SuRe™ MabSelect SuRe LX, MabSelect, MabSelect Xtra, rProtein A Sepharose from GE Healthcare, and ProSep HC, ProSep Ultra, and ProSep Ultra Plus from EMD Millipore.

In certain embodiments, the Protein A media is composed of chromatographic backbone with pendant protein ligands derived from Staphylococcus aureus. The Protein A ligand is linked either directly, or indirectly, to a variety of matrices including cross-linked agarose, polyacrylamide in ceramic macrobeads, porous glass, polystyrenedivenylbenzene, polymeric and polymethacrylate.

The Protein A column can be equilibrated with a suitable buffer prior to sample loading. In one embodiment, the equilibration buffer comprises a kosmotropic salt. For example, the kosmotropic salt may be a sulfate salt, a citrate salt, a phosphate salt, or a combination thereof. In a particular embodiment, the kosmotropic salt solution includes a salt selected from the group consisting of ammonium sulfate, sodium sulfate, sodium citrate, potassium sulfate, potassium phosphate, sodium phosphate or a combination thereof. In one embodiment, the kosmotropic salt is ammonium sulfate. In another embodiment, the kosmotropic salt is sodium sulfate. In yet another embodiment, the kosmotropic salt is sodium citrate.

In certain embodiments, the equilibration buffer salt has a concentration of between about 100 mM and 1500 mM. In yet another embodiment, the equilibration buffer has a pH between about 4.0 and 8.5 or between about 5.0 and 7.0. A non-limiting example of a suitable equilibration buffer is a Tris buffer at a pH of about 7.5. In one embodiment, the equilibration buffer is a Tris buffer including ammonium sulfate as a kosmotropic salt. Other non-limiting examples of suitable equilibration conditions are 20 mM Tris, pH of about 7.5, a PBS buffer, or 20 mM Tris, 1.1 M ammonium sulfate, pH 7.5 buffer.

Following equilibration of the chromatographic material, a sample containing the antibody, e.g., a non-human antibody, such as a canine antibody, and the at least one impurity is contacted to the chromatographic material in the presence of a load buffer to allow binding of a substantial portion of the antibody, while a substantial portion of the at least one impurity does not bind to the Protein A media.

In one embodiment, the load buffer comprises a kosmotropic salt, for example, a sulfate salt, a citrate salt, a phosphate salt, or a combination thereof. In a particular embodiment, the load buffer includes a kosmotropic salt solution having a salt selected from the group consisting of ammonium sulfate, sodium sulfate, sodium citrate, potassium sulfate, potassium phosphate, sodium phosphate or a combination thereof. In one embodiment, the kosmotropic salt is ammonium sulfate. In another embodiment, the kosmotropic salt is sodium sulfate. In yet another embodiment, the kosmotropic salt is sodium citrate.

In one embodiment, the load buffer and the equilibration buffer are the same. In another embodiment, the load buffer and the equilibration buffer are substantially the same. In yet another embodiment, the salt concentration and/or the pH of the load buffer are within about 50%, 40%, 30%, 20%, 15%, 10% or 5% of the salt concentration, and/or the pH of the equilibration buffer.

In certain embodiments, the load challenge of the sample comprising the antibody and at least one impurity is adjusted to a total protein load to the column of between about 10 and 100 g/L, or between about 20 and 80 g/L, or between about 30 and 60 g/L of Protein A media. In another embodiment, the load challenge is about 10 g, about 20 g, about 30 g, about 40 g, about 50 g, about 60 g, about 70 g, about 80 g, about 90 g, or about 100 g of the non-human antibody per one liter of Protein A media.

In another embodiment, the concentration of the at least one impurity in the sample is about 100 ng to about 300 ng per mg of antibody.

In one embodiment, the substantial portion of the antibody that binds to the Protein A media is at least about 20%, at least about 30%, at least about 40%, at least about 50%, at least about 60%, at least about 70%, at least about 80%, at least about 90%, at least about 95% or at least about 100% of the amount of the antibody in the sample.

In one embodiment, the substantial portion of the at least one impurity that does not bind to the Protein A media is at least about 20%, at least about 30%, at least about 40%, at least about 50%, at least about 60%, at least about 70%, at least about 80%, at least about 90%, at least about 95% or at least about 100% of the amount of the impurity in the sample.

The media is then subjected to a wash buffer, thereby allowing for a substantial portion of the at least one impurity that is not bound to the Protein A media, to flow past the Protein A media. The wash step may be performed one or more times.

In one embodiment, the wash buffer comprises a kosmotropic salt, for example, a sulfate salt, a citrate salt, a phosphate salt, or a combination thereof. In a particular embodiment, the wash buffer includes a kosmotropic salt solution having a salt selected from the group consisting of ammonium sulfate, sodium sulfate, sodium citrate, potassium sulfate, potassium phosphate, sodium phosphate or a combination thereof. In one embodiment, the kosmotropic salt is ammonium sulfate. In another embodiment, the kosmotropic salt is sodium sulfate. In yet another embodiment, the kosmotropic salt is sodium citrate.

In one embodiment, the wash buffer is the same as the load buffer and/or equilibration buffer. In another embodiment, the wash buffer is substantially the same as the load buffer and/or the equilibration buffer. In yet another embodiment, the salt concentration and/or the pH of the wash buffer are within about 50%, 40%, 30%, 20%, 15%, 10% or 5% of the salt concentration, and/or the pH of the load buffer and/or the equilibration buffer.

The Protein A media is then subjected to an elution buffer whereby the substantial portion of the antibody bound to the Protein A media is released from the Protein A media forming an elution fraction having a reduced level of the at least one impurity which is collected. In one embodiment of the invention, the elution buffer comprises Tris which has a concentration of about 5 min to about 100 mM. In another embodiment, the elution buffer has a pH of between about 5.0 to about 9.0. For example, a suitable elution buffer is an 0.1M acetic acid/NaCl buffer with a pH of about 3.5. Another example of a suitable elution buffer is a 20 mM Tris buffer with a pH of about 8.5.

According to the present invention, a substantial portion of the antibody reversibly binds to the Protein A media while a substantial portion of the at least one impurity flows past the Protein A media. The substantial portion of the antibody that binds to the Protein A media binds reversibly in that the bound antibody may be released therefrom under elution conditions, for example, by use of an elution buffer that comprising Tris at a pH of 8.5. The elution fraction(s) can be monitored using techniques well known to those skilled in the art. For example, the absorbance at OD₂₈₀ can be followed. Elution fractions can be collected starting with an initial deflection of about 0.5 AU to a reading of about 0.5 AU at the trailing edge of the elution peak. The elution fraction(s) of interest can then be prepared for further processing. For example, the collected sample can be titrated to a pH in the range of 5 to 8 using Tris buffer (e.g., 1.0 M) at a pH of about 10, and/or diluted to obtain a lower conductivity sample. Optionally, this titrated sample can be filtered and further processed.

In one embodiment, the substantial portion of the antibody released from the Protein A media upon elution with the elution buffer is at least about 20%, at least about 30%, at least about 40%, at least about 50%, at least about 60%, at least about 70%, at least about 80%, at least about 90% or about 100% of the amount of antibody bound to the Protein A media.

In another embodiment, the yield of the antibody in the elution fraction is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

Following contacting the sample with the Protein A media according to the method of the present invention, the elution fraction(s) includes the non-human antibody with a reduced level of the at least one impurity, e.g., host cell protein. In one embodiment of the invention, the elution fraction is substantially free of the at least one impurity, e.g., host cell protein. In another embodiment, the reduction of the at least one impurity in any one elution fraction is at least about 80%, at least about 90%, at least about 95%, at least about 98%, at least about 99%, or at least about 99.9%. In another embodiment, the at least one impurity is reduced by at least 0.25, at least 0.5, at least 0.75, at least 1.0, at least 1.25, at least 1.5, at least 2.0, at least 2.5, at least 3.0 or at least 3.5 log reduction fraction.

In various embodiments, the impurity is a process-related impurity. For example, the impurity may be a process-related impurity selected from the group consisting of a host cell protein, a host cell nucleic acid, a media component, and a chromatographic material. In a particular embodiment, the impurity is a host cell protein.

### Complementary Purification Techniques

In certain embodiments, a combination of Protein A and at least one of AEX (anion exchange chromatography) and CEX (cation exchange chromatography) and HIC (hydrophobic interaction chromatography) and MM (mixed-mode chromatography) methods can be used to prepare preparations of the antibody having a reduced level of impurity, including certain embodiments where one technology is used in a complementary/supplementary manner with another technology. In certain embodiments, such a combination can be performed such that certain sub-species are removed predominantly by a particularly technology, such that the combination provides the desired final composition/product quality. In certain embodiments, such combinations include the use of additional intervening chromatography, filtration, pH adjustment, UF/DF (ultrafiltration/diafiltration) steps so as to achieve the desired product quality, ion concentration, and/or viral reduction.

### Ion Exchange Chromatography

In certain embodiments, a precursor sample is subjected to ion exchange chromatography to purify the antibody, prior to the methods of the present invention. Alternatively or in addition, the elution fraction(s) generated by the methods of the present invention can be subjected to ion exchange chromatography to further purify the antibody. As noted above, certain embodiments of the present invention will employ one or more ion exchange chromatography steps prior to the Protein A purification step, while others will employ an ion exchange chromatography step after or both before and after the Protein A purification step.

As used herein, ion exchange separations includes any method by which two substances are separated based on the difference in their respective ionic charges, either on the antibody and/or chromatographic material as a whole or locally on specific regions of the antibody and/or chromatographic material, and thus can employ either cationic exchange material or anionic exchange material. For the purification of an antibody, the antibody must have a charge opposite to that of the functional group attached to the ion exchange material, e.g., media, in order to bind. For example, antibodies, which generally have an overall positive change in the buffer pH below its pI, will bind well to cation exchange material, which contain negatively charged functional groups.

The use of a cationic exchange material versus an anionic exchange material is based on the local charges of the antibody in a given solution. Therefore, it is within the scope of this invention to employ an anionic exchange step prior to the use of a Protein A step, or a cationic exchange step prior to the use of a Protein A step. Furthermore, it is within the scope of this invention to employ only a cationic exchange step, only an anionic exchange step, or any serial combination of the two either prior to or subsequent to the Protein A step.

In performing the separation, the sample containing the antibody (e.g., a non-human antibody such as a canine antibody) can be contacted with the ion exchange material by using any of a variety of techniques, e.g., using a batch purification technique or a chromatographic technique, as described above in connection with Protein A purification step.

In the context of batch purification, ion exchange material is prepared in, or equilibrated to, the desired starting buffer. Upon preparation, or equilibration, a slurry of the ion exchange material is obtained. The antibody solution is contacted with the slurry to adsorb the antibody to be separated to the ion exchange material. The solution comprising the at least one impurity (e.g., host cell proteins) that do not bind to the ion exchange material is separated from the slurry, e.g., by allowing the slurry to settle and removing the supernatant. The slurry can be subjected to one or more wash steps. If desired, the slurry can be contacted with a solution of higher conductivity to desorb the at least one impurity that have bound to the ion exchange material. In order to elute bound polypeptides (e.g., the antibody), the salt concentration of the buffer can be increased.

Alternatively, a packed ion-exchange chromatography column or an ion-exchange membrane device can be operated in a bind-elute mode, a flow-through, or a hybrid mode. In the bind-elute mode, the column or the membrane device is first conditioned with a buffer with a low ionic strength and proper pH under which the protein carries sufficient opposite change to that immobilized on the resin based matrix. During the feed load, the antibody will be adsorbed to the resin due to electrostatic attraction. After washing the column or the membrane device with the equilibration buffer or another buffer with different pH and/or conductivity, the product recovery is achieved by increasing the ionic strength (i.e., conductivity) of the elution buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution). In the flow-through mode, the column or the membrane device is operated at selected pH and conductivity such that the antibody does not bind to the resin or the membrane while the at least one impurity (e.g., host cell proteins, host cell nucleic acid, virus, aggregates) will be retained to the column or to the membrane. The column is then regenerated before next use.

Anionic or cationic substituents may be attached to matrices in order to form anionic or cationic supports for chromatography. Non-limiting examples of anionic exchange substituents include diethylaminoethyl (DEAE), quaternary aminoethyl (QAE) and quaternary amine (Q) groups. Cationic substituents include carboxymethyl (CM), sulfoethyl (SE), sulfopropyl (SP), phosphate (P) and sulfonate (S). Cellulose ion exchange medias such as DE23™, DE32™, DE52™, CM-23™, CM-32™, and CM-52™ are available from Whatman Ltd. Maidstone, Kent, U.K. SEPHADEX®-based and -locross-linked ion exchangers are also known. For example, DEAE-, QAE-, CM-, and SP-SEPHADEX® and DEAE-, Q-, CM- and S-SEPHAROSE® and SEPHAROSE® Fast Flow, and Capto™ S are all available from GE Healthcare. Further, both DEAE and CM derivitized ethylene glycol-methacrylate copolymer such as TOYOPEARL™ DEAE-6505 or M and TOYOPEARL™ CM-650S or M are available from Toso Haas Co., Philadelphia, Pa., or Nuvia S and UNOSphere™ S from BioRad, Hercules, Calif., Eshmuno® S from EMD Millipore, Billerica, Calif.

A mixture comprising an antibody (e.g., a non-human antibody, such as a canine antibody) and at least one impurity, e.g., HCP(s), is loaded onto an ion exchange column, such as an anion exchange column. For example, but not by way of limitation, the mixture can be loaded at a load level of about 40 g protein/L resin depending upon the column used. An example of a suitable anion exchange resin is Capto Q (GE Healthcare). The mixture loaded onto Capto Q column can be subsequently washed with wash buffer (equilibration buffer). The antibody is then eluted from the column, and a first eluate is obtained.

This ion exchange step facilitates the purification of the antibody by reducing impurities such as HCPs, host cell nucleic acids and aggregates. In certain aspects, the ion exchange column is an anion exchange column. For example, but not by way of limitation, a suitable resin for such an anion exchange column is Capto Q, Q Sepharose Fast Flow, and Poros HQ 50. These resins are available from commercial sources such as GE Healthcare and Life Technologies. This anion exchange chromatography process can be carried out at or around room temperature.

### Hydrophobic Interaction Chromatography

In certain embodiments, a precursor sample is subjected to hydrophobic interaction chromatography (HIC) to purify the antibody, prior to the methods of the present invention. Alternatively or in addition, the elution fraction(s) generated by the methods of the present invention can be subjected to HIC to further purify the antibody. As noted above, certain embodiments of the present invention will employ one or more HIC steps prior to the Protein A purification step, while others will employ a HIC step after or both before and after the Protein A purification step. The instant invention features methods for producing a preparation comprising an antibody (e.g., a non-human antibody, such as a canine antibody) having a reduced level of at least one impurity, for example, host cell proteins, from a sample comprising the antibody and at least one impurity by contacting the sample with Protein A media.

HIC purification of an antibody comprises reversible binding of the antibody and binding of one or more impurities through hydrophobic interaction with hydrophobic moieties attached to a solid matrix support (e.g., agarose). The hydrophobic interaction between molecules results from the tendency of a polar environment to exclude non-polar (i.e., hydrophobic) molecules. HIC relies on this principle of hydrophobicity of molecules (i.e., the tendency of a given protein to bind adsorptively to hydrophobic sites on a hydrophobic adsorbent body) to separate biomolecules based on their relative strength of interaction with the hydrophobic moieties (see, e.g., U.S. Pat. No. 4,000,098 and U.S. Pat. No. 3,917,527 which are herein incorporated by reference in their entirety). An advantage of this separation technique is its non-denaturing characteristics and the stabilizing effects of salt solutions used during loading, washing and or eluting.

Hydrophobic interaction chromatography employs the hydrophobic properties of molecules (e.g., proteins, polypeptides, lipids) to achieve separation of even closely-related molecules. Hydrophobic groups on the molecules interact with hydrophobic groups of the media or the membrane. In certain embodiments, the more hydrophobic a molecule is, the stronger it will interact with the column or the membrane. Thus, HIC purification, can be used to remove a variety of impurities, for example, process-related impurities (e.g., host cell proteins, DNA) as well as product-related species (e.g., high and low molecular weight product-related species, such as protein aggregates and fragments).

In performing the HIC separation, the sample is contacted with the HIC media, e.g., using a batch purification technique or using a column or membrane chromatography or monolithic material (referred to as HIC media or resin). For example, in the context of chromatographic separation, a chromatographic apparatus, commonly cylindrical in shape, is employed to contain the chromatographic support media (e.g., HIC media) prepared in an appropriate buffer solution. Once the chromatographic material is added to the chromatographic apparatus, a sample containing the antibody, and the at least one impurity is contacted to the chromatographic material in the presence of a loading buffer to allow binding of a portion of the antibody and a substantial portion of the impurity to the HIC media. A portion of the antibody in the sample binds to the HIC media while a portion of the antibody flows through, forming a flow through fraction having a reduced level of impurity which is collected.

The media is then subjected to a wash buffer, thereby allowing for a portion of the bound antibody to release from the HIC media in a wash fraction which is collected, while a substantial portion of the impurity remains bound to the HIC media. After loading, the column can be regenerated with water and cleaned with caustic solution to remove the bound impurities before next use.

In order to achieve the desired reversible binding of the antibody and the comparable strong binding of the at least one impurity, appropriate selection of resin, buffer, concentration, pH and sample load is required.

Hydrophobic interactions are strongest at high salt concentration (and hence the ionic strength of the anion and cation components). Adsorption of the antibody to a HIC column is favored by high salt concentrations, but the actual concentrations can vary over a wide range depending on the nature of the antibody, salt type and the particular HIC ligand chosen.

Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba²⁺; Ca²⁺; Mg²⁺; Li⁺; Cs⁺; Na⁺; K⁺; Rb⁺; NH₄⁺, while anions may be ranked in terms of increasing chaotropic effect as PO₄³⁻; SO₄²⁻; CH₃CO₃⁻; Cl⁻; Br⁻; NO₃⁻; ClO₄⁻; I⁻; SCN⁻.

In certain embodiments, the anionic part of the salt is chosen from among sulfate, citrate, chloride, or a mixture thereof. In certain embodiments, the cationic part of the salt is chosen from among ammonium, sodium, potassium, or a mixture thereof. In general, Na⁺, K⁺ or NH₄⁺ sulfates effectively promote ligand-protein interaction in HIC. Salts may be formulated that influence the strength of the interaction as given by the following relationship: (NH₄)₂SO₄>Na₂SO₄>NaCl>NH₄Cl>NaBr>NaSCN. In general, salt concentrations of between about 0.75 and about 2 M ammonium sulfate or between about 1 and 4 M NaCl are useful. In another embodiment, the load buffer and the wash buffer comprise a salt of the Hofmeister series or lyotropic series of salts.

In certain embodiments, the HIC adsorbent material is composed of a chromatographic backbone with pendant hydrophobic interaction ligands. For example, but not by way of limitation, the HIC media can be composed of convective membrane media with pendent hydrophobic interaction ligands, convective monolithic media with pendent hydrophobic interaction ligands, and/or convective filter media with embedded media containing the pendant hydrophobic interaction ligands.

In certain embodiments, the HIC adsorbent material can comprise a base matrix (e.g., derivatives of cellulose, polystyrene, synthetic poly amino acids, synthetic polyacrylamide gels, cross-linked dextran, cross-linked agarose, synthetic copolymer material or even a glass surface) to which hydrophobic ligands (e.g., alkyl, aryl and combinations thereof) are coupled or covalently attached using difunctional linking groups such as —NH—, —S—, —COO—, etc. The hydrophobic ligand may be terminated in a hydrogen but can also terminate in a functional group such as, for example, NH₂, SO₃H, PO₄H₂, SH, imidazoles, phenolic groups or non-ionic radicals such as OH and CONH₂. In one embodiment, the HIC media comprises at least one hydrophobic ligand. In another embodiment, the hydrophobic ligand is selected from the group consisting of butyl, hexyl, phenyl, octyl, or polypropylene glycol ligands.

One, non-limiting, example of a suitable HIC media comprises an agarose media or a membrane functionalized with phenyl groups (e.g., a Phenyl Sepharose™ from GE Healthcare or a Phenyl Membrane from Sartorius). Many HIC medias are available commercially. Examples include, but are not limited to, Tosoh Hexyl, CaptoPhenyl, Phenyl Sepharose™ 6 Fast Flow with low or high substitution, Phenyl Sepharose™ High Performance, Octyl Sepharose™ High Performance (GE Healthcare); Fractogel™ EMD Propyl or Fractogel™ EMD Phenyl (E. Merck, Germany); Macro-Prep™ Methyl or Macro-Prep™ t-Butyl columns (Bio-Rad, California); WP HI-Propyl (C3)™ (J. T. Baker, New Jersey); Toyopearl™ ether, phenyl or butyl (TosoHaas, PA); ToyoScreen PPG, ToyoScreen Phenyl, ToyoScreen Butyl, and ToyoScreen Hexyl are a rigid methacrylic polymer bead. GE HiScreen Butyl FF and HiScreen Octyl FF are high flow agarose based beads.

Because the pH selected for any particular purification process must be compatible with protein stability and activity, particular pH conditions may be specific for each application. A high or low pH may serve to weaken hydrophobic interactions and retention of proteins changes.

The pH of the HIC purification process is dependent, in part, on the pH of the buffers used to load, equilibrate and or wash the chromatographic resin or media.

In certain embodiments, HIC chromatographic fractions are collected during the load and/or wash cycles and are combined after appropriate analysis to provide an antibody preparation that contains the reduced level of impurities. In certain embodiments, the flow through fraction is combined with certain wash fractions to improve the yield of the process while still achieving the desired, e.g., reduced level of impurities in the preparation.

In certain embodiments, spectroscopy methods such as UV, NIR, FTIR, Fluorescence, Raman may be used to monitor levels of impurities such as aggregates and low molecular weight variants (e.g., fragments of the antibody) in an on-line, at-line or in-line mode, which can then be used to control the level of aggregates in the pooled material collected from the HIC methods of the present invention. In certain embodiments, on-line, at-line or in-line monitoring methods can be used either on the wash line of the chromatography step or in the collection vessel, to enable achievement of the desired product quality/recovery. In certain embodiments, the UV signal can be used as a surrogate to achieve an appropriate product quality/recovery, wherein the UV signal can be processed appropriately, including, but not limited to, such processing techniques as integration, differentiation, moving average, such that normal process variability can be addressed and the target product quality can be achieved. In certain embodiments, such measurements can be combined with in-line dilution methods such that ion concentration/conductivity of the load/wash can be controlled by feedback, thereby facilitating product quality control.

### Mixed Mode Chromatography

In certain embodiments, a precursor sample is subjected to mixed mode chromatography to purify the antibody, prior to the Protein A purification methods of the present invention. Alternatively or in addition, the elution fraction(s) generated by the methods of the present invention can be subjected to mixed mode chromatography to further purify the antibody. As noted above, certain embodiments of the present invention will employ one or more mixed mode chromatography steps prior to the Protein A purification step, while others will employ a mixed mode chromatography step after or both before and after the Protein A purification step.

Mixed mode chromatography is chromatography that utilizes a mixed mode media, such as, but not limited to CaptoAdhere available from GE Healthcare. Such a media comprises a mixed mode chromatography ligand. In certain embodiments, such a ligand refers to a ligand that is capable of providing at least two different, but co-operative, sites which interact with the substance to be bound. One of these sites gives an attractive type of charge-charge interaction between the ligand and the antibody. The other site typically gives electron acceptor-donor interaction and/or hydrophobic and/or hydrophilic interactions. Electron donor-acceptor interactions include interactions such as hydrogen-bonding, π-π, cation-π, charge transfer, dipole-dipole, induced dipole etc. The mixed mode functionality can give a different selectivity compared to traditional anion exchangers. For example, CaptoAdhere is designed for post-Protein A purification of monoclonal antibodies, where removal of leached Protein A, aggregates, host cell proteins, nucleic acids and viruses from monoclonal antibodies is performed in flow-through mode (the antibodies pass directly through the column while the contaminants are adsorbed). Mixed mode chromatography ligands are also known as “multimodal” chromatography ligands.

In certain embodiments, the mixed mode chromatography media is comprised of mixed mode ligands coupled to an organic or inorganic support, sometimes denoted a base matrix, directly or via a spacer. The support may be in the form of particles, such as essentially spherical particles, a monolith, filter, membrane, surface, capillaries, etc. In certain embodiments, the support is prepared from a native polymer, such as cross-linked carbohydrate material, such as agarose, agar, cellulose, dextran, chitosan, konjac, carrageenan, gellan, alginate etc. To obtain high adsorption capacities, the support can be porous, and ligands are then coupled to the external surfaces as well as to the pore surfaces. Such native polymer supports can be prepared according to standard methods, such as inverse suspension gelation (S. Hjerten, Biochim Biophys Acta 79(2), 393-398 (1964). Alternatively, the support can be prepared from a synthetic polymer, such as cross-linked synthetic polymers, e.g. styrene or styrene derivatives, divinylbenzene, acrylamides, acrylate esters, methacrylate esters, vinyl esters, vinyl amides etc. Such synthetic polymers can be produced according to standard methods, see e.g. “Styrene based polymer supports developed by suspension polymerization” (R. Arshady, Chimica e L'Industria 70(9), 70-75 (1988)). Porous native or synthetic polymer supports are also available from commercial sources, such as Amersham Biosciences, Uppsala, Sweden.

### Viral Inactivation

In certain embodiments, the elution fractions generated by the methods of the present invention can be subjected to viral inactivation to further purify the non-human antibody. A proper detergent concentration or pH and time can be selected to obtain desired viral inactivation results. After viral inactivation, the Protein A elution faction is usually pH and/or conductivity adjusted as necessary for further purification processes.

### Viral Filtration

In certain embodiments, a precursor sample is subjected to viral filtration to purify the antibody, prior to the Protein A purification methods of the present invention. Alternatively or in addition, the elution fractions generated by the methods of the present invention can be subjected to viral filtration to further purify the antibody. As noted above, certain embodiments of the present invention will employ one or more viral filtration steps prior to the Protein A purification step, while others will employ viral filtration after or both before and after the Protein A purification step.

Viral filtration is a dedicated viral reduction step in the entire purification process. This step is usually performed post chromatographic polishing steps. Viral reduction can be achieved via the use of suitable filters including, but not limited to, Planova 20N™, 50 N or BioEx from Asahi Kasei Pharma, Viresolve™ filters from EMD Millipore, ViroSart CPV from Sartorius, or Ultipor DV20 or DV50™ filter from Pall Corporation. It will be apparent to one of ordinary skill in the art to select a suitable filter to obtain desired filtration performance.

### Ultrafiltration/Diafiltration

In certain embodiments, a precursor sample is subjected to ultrafiltration and/or diafiltration to purify the antibody, prior to the Protein A purification methods of the present invention. Alternatively or in addition, the elution fraction(s) generated by the methods of the present invention can be subjected to ultrafiltration and/or diafiltration to further purify the antibody. As noted above, certain embodiments of the present invention will employ one or more ultrafiltration and/or diafiltration steps prior to the Protein A purification step, while others will employ ultrafiltration and/or diafiltration after or both before and after the Protein A purification step.

Certain embodiments of the present invention employ ultrafiltration and diafiltration steps to further concentrate and formulate the antibody product. Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). One filtration process is Tangential Flow Filtration as described in the Millipore catalogue entitled “Pharmaceutical Process Filtration Catalogue” pp. 177-202 (Bedford, Mass., 1995/96). Ultrafiltration is generally considered to mean filtration using filters with a pore size of smaller than 0.1 μm. By employing filters having such small pore size, the volume of the sample can be reduced through permeation of the sample buffer through the filter membrane pores while antibodies are retained above the membrane surface.

Diafiltration is a method of using membrane filters to remove and exchange salts, sugars, and non-aqueous solvents, to separate free from bound species, to remove low molecular-weight species, and/or to cause the rapid change of ionic and/or pH environments. Microsolutes are removed most efficiently by adding solvent to the solution being diafiltered at a rate approximately equal to the permeate flow rate. This washes away microspecies from the solution at a constant volume, effectively purifying the retained antibody. In certain embodiments of the present invention, a diafiltration step is employed to exchange the various buffers used in connection with the instant invention, optionally prior to further chromatography or other purification steps, as well as to remove impurities from the antibody preparations.

One of ordinary skill in the art can select appropriate membrane filter device for the UF/DF operation. Examples of membrane cassettes suitable for the present invention include, but not limited to, Pellicon 2 or Pellicon 3 cassettes with 10 kD, 30 kD or 50 kD membranes from EMD Millipore, Kvick 10 kD, 30 kD or 50 kD membrane cassettes from GE Healthcare, and Centramate or Centrasette 10 kD, 30 kD or 50 kD cassettes from Pall Corporation.

### Depth Filtration

In certain embodiments, a precursor sample is subjected to depth filtration to purify the antibody, prior to the Protein A purification methods of the present invention. Alternatively or in addition, the elution fraction(s) generated by the methods of the present invention can be subjected to depth filtration to further purify the antibody. As noted above, certain embodiments of the present invention will employ one or more depth filtration steps prior to the Protein A purification step, while others will employ depth filtration after or both before and after the Protein A purification step.

Depth filtration can serve to remove turbidity and/or various impurities from the non-human antibody prior to additional chromatography polishing steps. Examples of depth filters include, but not limited to, Millistak+X0HC, F0HC, D0HC, A1HC, and B1HC Pod filters (EMD Millipore), or Zeta Plus 30ZA/60ZA, 60ZA/90ZA, delipid, VR07, and VR05 filters (3M). In one embodiment, X0HC depth filter can be used to process the Protein A eluate before an ion-exchange chromatography step. The Protein A eluate pool may need to be conditioned to proper pH and conductivity to obtain desired impurity removal and product recovery from the depth filtration step.

### Exemplary Purification Strategies

Multiple process schemes based on the concepts of present invention can be employed to efficiently purify a MAb with weak binding strength for a Protein A chromatography media. Two non-limiting examples are described here for illustration purposes. Variation and modification of these examples, such as changing the order of one or more of the steps, are within the scope of this invention.

A Two-Column Purification Scheme

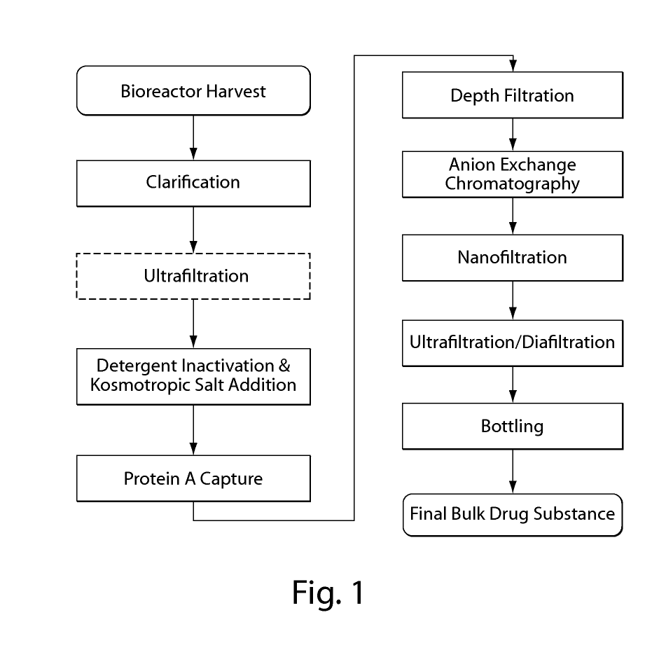

FIG. 1 depicts a two-column process for purification of a weak Protein A binding MAb. The harvest sample is first clarified to remove cells and cell debris using centrifugation, depth filtration, or the combination of both. If the clarified harvest, also known as the “primary recovery sample,” has an MAb titer less than about 1 g/L, it can be concentrated first by an ultrafiltration step to increase MAb concentration prior to further processing. The ultrafiltration is typically operated in the tangential flow filtration (or TFF) mode. The concentrated harvest can then be added with a detergent (e.g. 0.1% Tween 80 or Triton-X 100) to inactivate mammalian virus if present. The inactivated primary recovery harvest sample is then supplemented with a kosmotropic salt to obtain a conditioned primary recovery harvest sample with desired salt and protein concentration. The kosmotropic salt can be (NH₄)₂SO₄, Na₂SO₄, NaCitrate, K₂SO₄, K₃PO₄, Na₃PO₄, or a combination thereof. The MAb concentration in this conditioned primary recovery sample can range from about 1 g/L to about 10 g/L, while in certain embodiments the concentration is from about 1.5 g/L to about 8 g/L, about 1.5 g/L to about 5.8 g/L, about 1.7 g/L to about 5.8 g/L, about 1.9 g/L to about 5.45 g/L, about 1.9 g/L to about 4.95 g/L, about 1.9 g/L to about 4.7 g/L, about 1.9 g/L to about 4.5 g/L, or about 1.9 g/L to about 3.6 g/L. In certain embodiments, the concentration is about 1.5 g/L, about 1.9 g/L, about 3.6 g/L, about 4.5 g/L, about 4.7 g/L, about 4.95 g/L, about 5.45 g/L, or about 5.8 g/L. This material is usually filtered through a 0.2 um filter to remove any precipitates or turbidity formed during this process.

The conditioned and filtered primary recovery harvest sample is then subjected to a Protein A capture chromatography step. Any commercial Protein A resins or membranes can be employed here, including but not limited to, MabSelect SuRe, MabSelect SuRe LX, MabSelect, MabSelect Xtra from GE Healthcare, and ProSep HC, ProSep Ultra Plus, and ProSep Ultra Plus from EMD Millipore. The equilibration buffer contains the same concentrations of the komotropic salt as that used in the load material. One or multiple wash steps can be performed to reduce impurities such as HCPs. These wash buffers may contain the same concentrations of komotropic salt as used in the load, or higher or lower concentrations. In certain embodiments, a higher salt buffer was used in the first wash step followed by the equilibration buffer wash. An example of a suitable equilibration buffer is a Tris buffer with pH of about 6 to 8, or, in certain embodiments, about 7.5, containing a komotropic salt. A specific example of suitable equilibration is 20 mM Tris, 0.5 M (NH₄)₂SO₄, pH 7.5, wash 1 buffer is 20 mM Tris, 0.8 M (NH₄)₂SO₄, pH 7.5, and wash 2 buffer is the same as equilibration buffer. The Protein A column elution can be achieved using either a low pH or a high pH buffer. An example of high pH buffer is 20 mM Tris, pH 8.5 buffer. The eluate can be monitored using techniques well known to those skilled in the art. For example, the absorbance at UV₂₈₀ can be followed. The eluate can be collected starting with an initial deflection of about 500 mAU to a reading of about 500 mAU at the trailing edge of the elution peak. The elution fraction(s) of interest can then be prepared for further processing.

The Protein A eluate can be pH and/or conductivity adjusted to a target condition prior to fine purification. An example of such condition is pH 8 and about 28 mS/cm. A depth filtration step can be used to remove any precipitate or turbidity formed during this conditioning step; it also reduces impurities including HCP, aggregates, DNA, and leach Protein A. In certain embodiments, the depth filter is Millistak+X0HC Pod filter (EMD Millipore). Other filters with cationic charge functionality can also be used in this step.

The depth filtrate can then be purified through an anion exchange (AEX) chromatography step to further remove various impurities. Either AEX resin or AEX membrane can be used for this operation. An example of AEX resin is Capto Q or Q Sepharose Fast Flow (GE Healthcare). Either bind-elute or flow-through mode can be used for this step. In certain embodiments, Capto Q column was operated in the bind-elute mode to achieve desired product purity.

The AEX eluate is then processed through a viral filtration step to ensure sufficient viral removal for the overall process. Selecting a suitable viral filter can be performed by anyone skilled in the art. An example of suitable viral filter is Planova 20 N or BioEx from Asahi.

The viral filtrate is subjected to final ultrafiltration and diafiltration to formulate the antibody product. Commercial filters are available to effectuate this step. For example, a Biomax 30 kD membrane cassette (EMD Millipore) can be used to complete this step. The final product is then filled into proper containers before storage.

A Three-Column Purification Scheme

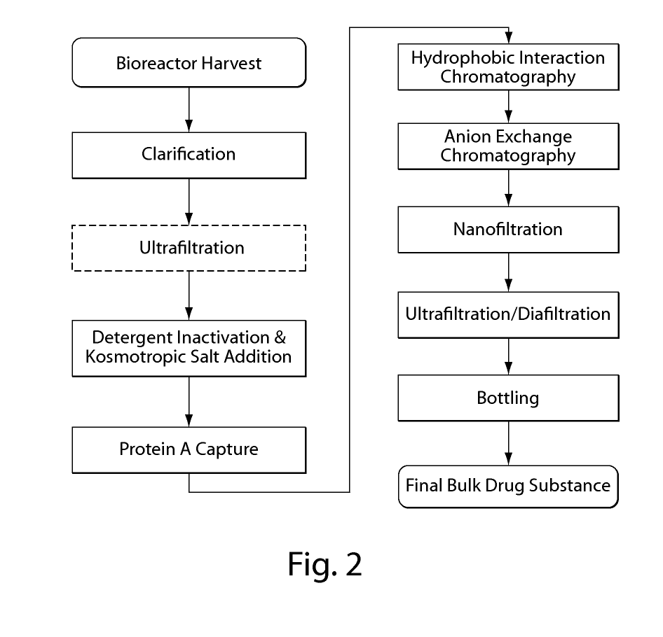

FIG. 2 shows a three-column process for purification of a weak Protein A binding MAb molecule. The key difference between this process and the two-column process is that a HIC chromatography step is used prior to the AEX polishing. When there is no significant precipitate or turbidity in the conditioned Protein A eluate, it can be processed directly through a HIC step first to remove HCP, DNA, aggregates and leached Protein A. This HIC step can be run in either flow-through or bind-elute mode, and can be a resin or a membrane. In some embodiments, Capto Phenyl resin is used and is run in the flow-through mode (GE Healthcare). The column is equilibrated with 20 mM Tris, 0.1 M (NH₄)₂SO₄, pH 7.5 buffer, then loaded with conditioned Protein A eluate at pH 7.5 and conductivity ˜23 mS/cm, and finally washed with the equilibration buffer again to recover the residual product retained within the column. The column may be loaded to 80 g/L of antibody, and the flow-through pool is collected during the load when UV280 reading reached 200 mAU and stopped during the wash when UV280 reading dropped back to 200 mAU. The HIC eluate is then processed through AEX chromatography to further purify the antibody to desired final purity. All the other steps are similar to those described in the two-column process scheme.

In the case of significant precipitate or turbidity is formed during the conditioning of the Protein A eluate, a depth filtration step can be used before the HIC chromatography. In this case, any depth filter that can remove particulates may be employed here.

In addition to the two exemplary process schemes described above, the cation exchange chromatography (CEX) step can be used in combination with a depth filtration, AEX or HIC step after the Protein A capture step to polish the antibody process stream. The viral inactivation step, if not performed prior to the Protein A capture step, can be done after the Protein A but before depth filtration and other chromatographic fine purifications operations.

Certain embodiments of the present invention will include further purification steps. Examples of additional purification procedures which can be performed prior to, during, or following the ion exchange chromatography method include ethanol precipitation, isoelectric focusing, reverse phase HPLC, chromatography on silica, chromatography on heparin Sepharose™, further anion exchange chromatography and/or further cation exchange chromatography, chromatofocusing, SDS-PAGE, ammonium sulfate precipitation, hydroxylapatite chromatography, gel electrophoresis, dialysis, and affinity chromatography (e.g., using protein G, an antibody, a specific substrate, ligand or antigen as the capture reagent).

### Methods of Assaying Sample Purity

### Assaying Host Cell Protein

The present invention also provides methods for determining the residual levels of host cell protein (HCP) concentration in the precursor sample or the elution fraction(s) following the Protein A steps of the present invention. As described above, HCPs are desirably excluded from the final target substance product. Exemplary HCPs include proteins originating from the source of the antibody production. Failure to identify and sufficiently remove HCPs from the target antibody may lead to reduced efficacy and/or adverse subject reactions. Accordingly, in one embodiment, the present invention further comprises assaying the sample for the level of host cell protein concentration prior to performing protein A chromatography. Alternatively or in combination, in certain embodiments, the present invention further comprises assaying the elution fraction for the level of host cell protein concentration following protein A chromatography.

As used herein, the term “HCP ELISA” refers to an ELISA where the second antibody used in the assay is specific to the HCPs produced from cells, e.g., CHO cells, used to generate the antibody. The second antibody may be produced according to conventional methods known to those of skill in the art. For example, the second antibody may be produced using HCPs obtained by sham production and purification runs, i.e., the same cell line used to produce the antibody is used, but the cell line is not transfected with antibody DNA. In an exemplary embodiment, the second antibody is produced using HCPs similar to those expressed in the cell expression system of choice, i.e., the cell expression system used to produce the target antibody.

Generally, HCP ELISA comprises sandwiching a liquid sample comprising HCPs between two layers of antibodies, i.e., a first antibody and a second antibody. The sample is incubated during which time the HCPs in the sample are captured by the first antibody, for example, but not limited to goat anti-CHO, affinity purified (Cygnus). A labeled second antibody, or blend of antibodies, specific to the HCPs produced from the cells used to generate the antibody, e.g., anti-CHO HCP Biotinylated, is added, and binds to the HCPs within the sample. In certain embodiments the first and second antibodies are polyclonal antibodies. In certain aspects the first and second antibodies are blends of polyclonal antibodies raised against HCPs. The amount of HCP contained in the sample is determined using the appropriate test based on the label of the second antibody.

HCP ELISA may be used for determining the level of HCPs in an antibody composition, such as an eluate or flow-through obtained using the process described above. The present invention also provides a composition comprising an antibody, wherein the composition has no detectable level of HCPs as determined by an HCP Enzyme Linked Immunosorbent Assay (“ELISA”).

Spectroscopy methods such as UV, NIR, FTIR, Fluorescence, Raman may be used to monitor levels of impurities such as host cell proteins in an on-line, at-line or in-line mode, which can then be used to control the level of host cell proteins in the material collected from the Protein A methods of the present invention. In certain embodiments, on-line, at-line or in-line monitoring methods can be used in the collection vessel, to enable achievement of the desired product quality/recovery. In certain embodiments, the UV signal can be used as a surrogate to achieve an appropriate product quality/recovery, wherein the UV signal can be processed appropriately, including, but not limited to, such processing techniques as integration, differentiation, moving average, such that normal process variability can be addressed and the target product quality can be achieved. In certain embodiments, such measurements can be combined with in-line dilution methods such that ion concentration/conductivity of the load/wash can be controlled by feedback, thereby facilitating product quality control.

### Assaying Affinity Chromatographic Material

In certain embodiments, the present invention also provides methods for determining the residual levels of affinity chromatographic material (e.g. protein A ligand) in the elution fraction(s). In certain contexts such material leaches into the antibody composition during the purification process. In certain embodiments, an assay for identifying the concentration of Protein A in the elution fraction(s) is employed. As used herein, the term “Protein A ELISA” refers to an ELISA where the second antibody used in the assay is specific to the Protein A employed to purify the antibody. The second antibody may be produced according to conventional methods known to those of skill in the art. For example, the second antibody may be produced using naturally occurring or recombinant Protein A in the context of conventional methods for antibody generation and production.

Generally, Protein A ELISA comprises sandwiching a liquid sample comprising Protein A (or possibly containing Protein A) between two layers of anti-Protein A antibodies, i.e., a first anti-Protein A antibody and a second anti-Protein A antibody. The sample is exposed to a first layer of anti-Protein A antibody, for example, but not limited to polyclonal antibodies or blends of polyclonal antibodies, and incubated for a time sufficient for Protein A in the sample to be captured by the first antibody. A labeled second antibody, for example, but not limited to polyclonal antibodies or blends of polyclonal antibodies, specific to the Protein A is then added, and binds to the captured Protein A within the sample. Additional non-limiting examples of anti-Protein A antibodies useful in the context of the instant invention include chicken anti-Protein A and biotinylated anti-Protein A antibodies. The amount of Protein A contained in the sample is determined using the appropriate test based on the label of the second antibody. Similar assays can be employed to identify the concentration of alternative affinity chromatographic materials.

Protein A ELISA may be used for determining the level of Protein A in an antibody composition, such as an eluate or flow-through obtained using the process described in above. The present invention also provides a composition comprising an antibody, wherein the composition has no detectable level of Protein A as determined by a Protein A Enzyme Linked Immunosorbent Assay (“ELISA”).

### Assaying Aggregates

In certain embodiments, the levels of product-related substances, such as aggregates, in either the initial sample or the elution fraction(s) following the Protein A steps of the present invention are analyzed. For example, but not by way of limitation, the aggregates present in the process samples can be quantified according to the following methods.

Aggregates may be measured using a size exclusion chromatographic (SEC) method whereby molecules are separated based on size and/or molecular weight such that larger molecules elute earlier from the column. For example, but not by way of limitation, a SEC columns useful for the detection of aggregates include: TSK-gel G3000SW×L, 5 μm, 125 Å, 7.8×300 mm column (Tosoh Bioscience), TSK-gel Super SW3000, 4 μm, 250 Å, 4.6×300 mm column (Tosoh Bioscience), or Zorbax GF450 column (Agilent Technologies). A further example of an SEC column for analysis of monomers and aggregates is the MAbPac™ SEC-1 (Thermo Scientific) column which may be used under non-denaturing conditions, in both high- and low-salt mobile phases, and with volatile eluents. In certain embodiments, the aforementioned columns are used along with an Agilent or a Shimazhu HPLC system. In a particular embodiment of SEC, aggregates may be quantified using a Zorbax GF450 column on an Agilent HPLC system.

In certain embodiments, sample injections are made under isocratic elution conditions using a mobile phase consisting of, for example, 100 mM sodium sulfate and 100 mM sodium phosphate at pH 6.8, and detected with UV absorbance at 214 nm. In certain embodiments, the mobile phase will consist of 1×PBS at pH 7.4, and elution profile detected with UV absorbance at 280 nm.

The elution profile may be further analyzed using multiangle laser light-scattering (MALS), to determine the apparent molecular weight of each peak, and allow identification as a dimer, tetramer, or other high molecular weight species. The elution profile may also be further analyzed using sodium dodecyl sulfate-polyacrylamide gel electrophoresis (SDS-PAGE). For example, the fraction is mixed with either a non-reducing or reducing denaturing sample buffer, treated for two minutes at 98° C. in an Eppendorf Thermomixer Contort, then loaded in a 5% polyacrylamide tris-HCL gel alongside pre-stained broad range molecular weight markers. Electrophoresis is performed using a buffer comprising 0.3% (w/v) Tris, 1.44% (w/v) glycine and 0.1% SDS, pH 8.3. Separation is performed at a constant current of 100 V and at maximally 50 mA for about 1 hour, followed by staining of the gel. In another embodiment, the aggregates may be analyzed and the molecular weight determined using high performance-size exclusion chromatography followed by native electrospray ionization time-of-flight mass spectrometry (ESI-TOF MS). Further methods for assaying levels of aggregates are provided in the Examples below.

### Assaying Charge and Size Variants

In certain embodiments, the levels of product-related substances, such as acidic species and other charge variants, in the chromatographic samples produced using the techniques described herein are analyzed. For example, but not by way of limitation, the acidic species and other charge variants present in the process samples can be quantified according to the following methods. Cation exchange chromatography was performed on a Dionex ProPac WCX-10, Analytical column 4 mm×250 mm (Dionex, CA). An Agilent 1200 HPLC system was used as the HPLC. The mobile phases used were 10 min Sodium Phosphate dibasic pH 7.5 (Mobile phase A) and 10 min Sodium Phosphate dibasic, 500 min Sodium Chloride pH 5.5 (Mobile phase B). A binary gradient (94% A, 6% B: 0-20 min; 84% A, 16% B: 20-22 min; 0% A, 100% B: 22-28 min; 94% A, 6% B: 28-34 min) was used with detection at 280 nm.

In certain embodiments, the levels of aggregates, monomer, and fragments in the chromatographic samples produced using the techniques described herein are analyzed. In certain embodiments, the aggregates, monomer, and fragments are measured using a size exclusion chromatographic (SEC) method for each molecule. For example, but not by way of limitation, a TSK-gel G3000SW×L, 5 μm, 125 Å, 7.8×300 mm column (Tosoh Bioscience) can be used in connection with certain embodiments, while a TSK-gel Super SW3000, 4 μm, 250 Å, 4.6×300 mm column (Tosoh Bioscience) can be used in alternative embodiments. In certain embodiments, the aforementioned columns are used along with an Agilent or a Shimazhu HPLC system. In certain embodiments, sample injections are made under isocratic elution conditions using a mobile phase consisting of, for example, 100 mM sodium sulfate and 100 mM sodium phosphate at pH 6.8, and detected with UV absorbance at 214 nm. In certain embodiments, the mobile phase will consist of 1×PBS at pH 7.4, and elution profile detected with UV absorbance at 280 nm. In certain embodiments, quantification is based on the relative area of detected peaks.

### Antibody Generation

Antibodies to be purified by the methods of the present invention can be generated by a variety of techniques, including immunization of an animal with the antigen of interest followed by conventional monoclonal antibody methodologies e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

In certain embodiments, the animal system for preparing hybridomas is the murine system. Hybridoma production is a well-established procedure Immunization protocols and techniques for isolation of immunized splenocytes for fusion are known in the art. Fusion partners (e.g., murine myeloma cells) and fusion procedures are also known.

In certain non-limiting embodiments, the antibodies of this disclosure are those having a weak binding strength for Protein A. In certain embodiments, the antibodies are feline monoclonal antibodies. In certain embodiments, the antibodies are canine monoclonal antibodies. In other embodiments, the antibodies are equine monoclonal antibodies. In yet another embodiment, the antibodies are murine antibodies, rat, bovine antibodies or other non-human antibodies.

An antibody can be, in certain embodiments, a chimeric antibody. DNA encoding the heavy and light chain immunoglobulins can be obtained from the non-human hybridoma of interest and engineered to contain non-murine immunoglobulin sequences using standard molecular biology techniques. For example, to create a chimeric antibody, murine variable regions can be linked to constant regions from other species using methods known in the art (see e.g., U.S. Pat. No. 4,816,567 to Cabilly et al.).

The antibodies or antigen-binding portions thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see, e.g., Canfield and Morrison (1991) J. Exp. Med. 173:1483-1491; and Lund et al. (1991) J. of Immunol. 147:2657-2662, the entire teachings of which are incorporated herein). Reduction in FcR binding ability of the antibody may also reduce other effector functions which rely on FcR interactions, such as opsonization and phagocytosis and antigen-dependent cellular cytotoxicity.

### Antibody Production

To express an antibody of the invention, DNAs encoding partial or full-length light and heavy chains are inserted into one or more expression vector such that the genes are operatively linked to transcriptional and translational control sequences. (See, e.g., U.S. Pat. No. 6,914,128, the entire teaching of which is incorporated herein by reference.) In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into a separate vector or, more typically, both genes are inserted into the same expression vector. The antibody genes are inserted into an expression vector by standard methods (e.g., ligation of complementary restriction sites on the antibody gene fragment and vector, or blunt end ligation if no restriction sites are present). Prior to insertion of the antibody or antibody-related light or heavy chain sequences, the expression vector may already carry antibody constant region sequences. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, a recombinant expression vector of the invention can carry one or more regulatory sequence that controls the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, e.g., in Goeddel; Gene Expression Technology: Methods in Enzymology 185, Academic Press, San Diego, Calif. (1990), the entire teaching of which is incorporated herein by reference. It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al. and U.S. Pat. No. 4,968,615 by Schaffner et al., the entire teachings of which are incorporated herein by reference.

In addition to the antibody chain genes and regulatory sequences, a recombinant expression vector of the invention may carry one or more additional sequences, such as a sequence that regulates replication of the vector in host cells (e.g., origins of replication) and/or a selectable marker gene. The selectable marker gene facilitates selection of host cells into which the vector has been introduced (see e.g., U.S. Pat. Nos. 4,399,216, 4,634,665 and 5,179,017, all by Axel et al., the entire teachings of which are incorporated herein by reference). For example, typically the selectable marker gene confers resistance to drugs, such as G418, hygromycin or methotrexate, on a host cell into which the vector has been introduced. Suitable selectable marker genes include the dihydrofolate reductase (DHFR) gene (for use in dhfr-host cells with methotrexate selection/amplification) and the neo gene (for G418 selection).

An antibody of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989) and in U.S. Pat. Nos. 4,816,397 & 6,914,128, the entire teachings of which are incorporated herein.

For expression of the light and heavy chains, the expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, such as mammalian host cells, is suitable because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss and Wood (1985) Immunology Today 6:12-13, the entire teaching of which is incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Suitable mammalian host cells for expressing the recombinant antibodies of the invention include Chinese Hamster Ovary (CHO cells) (including dhfr-CHO cells, described in Urlaub and Chasin, (1980) PNAS USA 77:4216-4220, used with a DHFR selectable marker, e.g., as described in Kaufman and Sharp (1982) Mol. Biol. 159:601-621, the entire teachings of which are incorporated herein by reference), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Other examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), the entire teachings of which are incorporated herein by reference.

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce an antibody may be cultured in a variety of media. Commercially available media such as Ham's F10™ (Sigma), Minimal Essential Medium™ ((MEM), (Sigma), RPMI-1640 (Sigma), and Dulbecco's Modified Eagle's Medium™ ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells, the entire teachings of which are incorporated herein by reference. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Host cells can also be used to produce portions of intact antibodies, such as Fab fragments or scFv molecules. It is understood that variations on the above procedure are within the scope of the present invention. For example, in certain embodiments it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to the antigen to which the putative antibody binds. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the one to which the putative antibody binds, depending on the specificity of the antibody of the invention, by crosslinking an antibody of the invention to a second antibody by standard chemical crosslinking methods.

In a suitable system for recombinant expression of an antibody of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

When using recombinant techniques, the antibody can be produced intracellularly, in the periplasmic space, or directly secreted into the medium. In one aspect, if the antibody is produced intracellularly, as a first step, the particulate debris, either host cells or lysed cells (e.g., resulting from homogenization), can be removed, e.g., by centrifugation or ultrafiltration. Where the antibody is secreted into the medium, supernatants from such expression systems can be first concentrated using a commercially available protein concentration filter, e.g., an Amicon™ or Millipore Pellicon™ ultrafiltration unit.

Prior to the process of the invention, procedures for purification of antibodies from cell debris initially depend on the site of expression of the antibody. Some antibodies can be secreted directly from the cell into the surrounding growth media; others are made intracellularly. For the latter antibodies, the first step of a purification process typically involves: lysis of the cell, which can be done by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. Where the antibody is secreted, supernatants from such expression systems are generally first concentrated using a commercially available protein concentration filter, e.g., an Amicon™ or Millipore Pellicon™ ultrafiltration unit. Where the antibody is secreted into the medium, the recombinant host cells can also be separated from the cell culture medium, e.g., by tangential flow filtration. Antibodies can be further recovered from the culture medium using the antibody purification methods of the invention.

### Methods of Treatment Using the Low Impurity Compositions of the Invention

The low impurity compositions, for example, low host cell protein compositions, of the invention may be used to treat any disorder in a subject, for example, a non-human subject for which the therapeutic antibody (e.g., a non-human antibody, such as a canine antibody) comprised in the composition is appropriate for treating.

A “disorder” is any condition that would benefit from treatment with the non-human antibody. This includes chronic and acute disorders or diseases including those pathological conditions which predispose the subject to the disorder in question.

As used herein, the term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the invention, the subject is a non-human subject.

As used herein, the term “treatment” or “treat” refers to both therapeutic treatment and prophylactic or preventative measures. Those in need of treatment include those already with the disorder, as well as those in which the disorder is to be prevented.

The low impurity compositions can be administered by a variety of methods known in the art. Exemplary routes/modes of administration include subcutaneous injection, intravenous injection or infusion. In certain aspects, a low impurity compositions may be orally administered. As will be appreciated by the skilled artisan, the route and/or mode of administration will vary depending upon the desired results.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. In certain embodiments it is especially advantageous to formulate parenteral compositions in dosage unit form for ease of administration and uniformity of dosage. Dosage unit form as used herein refers to physically discrete units suited as unitary dosages for the mammalian subjects to be treated; each unit comprising a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a low impurity composition of the invention is 0.01-20 mg/kg, or 1-10 mg/kg, or 0.3-1 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### Pharmaceutical Formulations Containing the Low Impurity Compositions of the Invention

The present invention further provides preparations and formulations comprising low impurity compositions, for example, low host cell protein compositions, of the invention. It should be understood that any antibody of interest, such as a non-human antibody, such as a canine antibody, may be formulated or prepared as described below. When various formulations are described in this section as including an antibody, such as a non-human antibody (e.g., canine antibody), it is understood that such an antibody may be an antibody having any one or more of the characteristics of the antibodies of interest described herein.

In certain embodiments, the low impurity compositions, for example, low host cell protein compositions, of the invention may be formulated with a pharmaceutically acceptable carrier as pharmaceutical (therapeutic) compositions, and may be administered by a variety of methods known in the art. As will be appreciated by the skilled artisan, the route and/or mode of administration will vary depending upon the desired results. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the antibodies of interest (e.g., a non-human antibody, such as a canine antibody) of the present invention, and with each other, in a manner such that there is no interaction which would substantially impair the desired pharmaceutical efficacy.

The low impurity compositions, for example, low host cell protein compositions, of the invention are present in a form known in the art and acceptable for therapeutic uses. In one embodiment, a formulation of the low impurity compositions, for example, low host cell protein compositions, of the invention is a liquid formulation. In another embodiment, a formulation of the low impurity compositions, for example, low host cell protein compositions, of the invention is a lyophilized formulation. In a further embodiment, a formulation of the low impurity compositions, for example, low host cell protein compositions, of the invention is a reconstituted liquid formulation. In one embodiment, a formulation of the low impurity compositions, for example, low host cell protein compositions, of the invention is a stable liquid formulation. In one embodiment, a liquid formulation of the low impurity compositions, for example, low host cell protein compositions, of the invention is an aqueous formulation. In another embodiment, the liquid formulation is non-aqueous. In a specific embodiment, a liquid formulation of the low impurity compositions, for example, low host cell protein compositions, of the invention is an aqueous formulation wherein the aqueous carrier is distilled water.

The formulations of the low impurity compositions, for example, low host cell protein compositions, of the invention comprise an antibody (e.g., a non-human antibody, such as a canine antibody) in a concentration resulting in a w/v appropriate for a desired dose. The antibody may be present in the formulation at a concentration of about 1 mg/ml to about 500 mg/ml, e.g., at a concentration of at least 1 mg/ml, at least 5 mg/ml, at least 10 mg/ml, at least 15 mg/ml, at least 20 mg/ml, at least 25 mg/ml, at least 30 mg/ml, at least 35 mg/ml, at least 40 mg/ml, at least 45 mg/ml, at least 50 mg/ml, at least 55 mg/ml, at least 60 mg/ml, at least 65 mg/ml, at least 70 mg/ml, at least 75 mg/ml, at least 80 mg/ml, at least 85 mg/ml, at least 90 mg/ml, at least 95 mg/ml, at least 100 mg/ml, at least 105 mg/ml, at least 110 mg/ml, at least 115 mg/ml, at least 120 mg/ml, at least 125 mg/ml, at least 130 mg/ml, at least 135 mg/ml, at least 140 mg/ml, at least 150 mg/ml, at least 200 mg/ml, at least 250 mg/ml, or at least 300 mg/ml.

In a specific embodiment, a formulation of the low impurity compositions, for example, low host cell protein compositions, of the invention comprises at least about 100 mg/ml, at least about 125 mg/ml, at least 130 mg/ml, or at least about 150 mg/ml of antibody (e.g., a non-human antibody, such as a canine antibody) of the invention.

In one embodiment, the concentration of antibody (e.g., a non-human antibody, such as a canine antibody), which is included in the formulation of the invention, is between about 1 mg/ml and about 25 mg/ml, between about 1 mg/ml and about 200 mg/ml, between about 25 mg/ml and about 200 mg/ml, between about 50 mg/ml and about 200 mg/ml, between about 75 mg/ml and about 200 mg/ml, between about 100 mg/ml and about 200 mg/ml, between about 125 mg/ml and about 200 mg/ml, between about 150 mg/ml and about 200 mg/ml, between about 25 mg/ml and about 150 mg/ml, between about 50 mg/ml and about 150 mg/ml, between about 75 mg/ml and about 150 mg/ml, between about 100 mg/ml and about 150 mg/ml, between about 125 mg/ml and about 150 mg/ml, between about 25 mg/ml and about 125 mg/ml, between about 50 mg/ml and about 125 mg/ml, between about 75 mg/ml and about 125 mg/ml, between about 100 mg/ml and about 125 mg/ml, between about 25 mg/ml and about 100 mg/ml, between about 50 mg/ml and about 100 mg/ml, between about 75 mg/ml and about 100 mg/ml, between about 25 mg/ml and about 75 mg/ml, between about 50 mg/ml and about 75 mg/ml, or between about 25 mg/ml and about 50 mg/ml.

In a specific embodiment, a formulation of the low impurity compositions, for example, low host cell protein compositions, of the invention comprises between about 90 mg/ml and about 110 mg/ml or between about 100 mg/ml and about 210 mg/ml of a antibody (e.g., a non-human antibody, such as a canine antibody).

The formulations of the low impurity compositions, for example, low host cell protein compositions, of the invention comprising an antibody (e.g., a non-human antibody, such as a canine antibody) may further comprise one or more active compounds as necessary for the particular indication being treated, typically those with complementary activities that do not adversely affect each other. Such additional active compounds are suitably present in combination in amounts that are effective for the purpose intended.

The formulations of the low impurity compositions, for example, low host cell protein compositions, of the invention may be prepared for storage by mixing the antibody (e.g., a non-human antibody, such as a canine antibody) having the desired degree of purity with optional physiologically acceptable carriers, excipients or stabilizers, including, but not limited to buffering agents, saccharides, salts, surfactants, solubilizers, polyols, diluents, binders, stabilizers, salts, lipophilic solvents, amino acids, chelators, preservatives, or the like (Goodman and Gilman's The Pharmacological Basis of Therapeutics, 12^{th} edition, L. Brunton, et al. and Remington's Pharmaceutical Sciences, 16th edition, Osol, A. Ed. (1999)), in the form of lyophilized formulations or aqueous solutions at a desired final concentration. Acceptable carriers, excipients, or stabilizers are nontoxic to recipients at the dosages and concentrations employed, and include buffers such as histidine, phosphate, citrate, glycine, acetate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrolidone; amino acids such as glycine, glutamine, asparagine, histidine, arginine, or lysine; monosaccharides, disaccharides, and other carbohydrates including trehalose, glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, polysorbate 80, PLURONICS™ or polyethylene glycol (PEG).

The buffering agent may be histidine, citrate, phosphate, glycine, or acetate. The saccharide excipient may be trehalose, sucrose, mannitol, maltose or raffinose. The surfactant may be polysorbate 20, polysorbate 40, polysorbate 80, or Pluronic F68. The salt may be NaCl, KCl, MgCl₂, or CaCl₂

The formulations of the low impurity compositions, for example, low host cell protein compositions, of the invention may include a buffering or pH adjusting agent to provide improved pH control. A formulation of the invention may have a pH of between about 3.0 and about 9.0, between about 4.0 and about 8.0, between about 5.0 and about 8.0, between about 5.0 and about 7.0, between about 5.0 and about 6.5, between about 5.5 and about 8.0, between about 5.5 and about 7.0, or between about 5.5 and about 6.5. In a further embodiment, a formulation of the invention has a pH of about 3.0, about 3.5, about 4.0, about 4.5, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.5, about 8.0, about 8.5, or about 9.0. In a specific embodiment, a formulation of the invention has a pH of about 6.0. One of skill in the art understands that the pH of a formulation generally should not be equal to the isoelectric point of the particular antibody (e.g., a non-human antibody, such as a canine antibody) to be used in the formulation.

Typically, the buffering agent is a salt prepared from an organic or inorganic acid or base. Representative buffering agents include, but are not limited to, organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. In addition, amino acid components can also function in a buffering capacity. Representative amino acid components which may be utilized in the formulations of the invention as buffering agents include, but are not limited to, glycine and histidine. In certain embodiments, the buffering agent is chosen from histidine, citrate, phosphate, glycine, and acetate. In a specific embodiment, the buffering agent is histidine. In another specific embodiment, the buffering agent is citrate. In yet another specific embodiment, the buffering agent is glycine. The purity of the buffering agent should be at least 98%, or at least 99%, or at least 99.5%. As used herein, the term “purity” in the context of histidine and glycine refers to chemical purity of histidine or glycine as understood in the art, e.g., as described in The Merck Index, 13^{th} ed., O'Neil et al. ed. (Merck & Co., 2001).

Buffering agents are typically used at concentrations between about 1 min and about 200 min or any range or value therein, depending on the desired ionic strength and the buffering capacity required. The usual concentrations of conventional buffering agents employed in parenteral formulations can be found in: Pharmaceutical Dosage Form: Parenteral Medications, Volume 1, 2^{nd} Edition, Chapter 5, p. 194, De Luca and Boylan, “Formulation of Small Volume Parenterals”, Table 5: Commonly used additives in Parenteral Products. In one embodiment, the buffering agent is at a concentration of about 1 min, or of about 5 min, or of about 10 min, or of about 15 min, or of about 20 min, or of about 25 min, or of about 30 min, or of about 35 min, or of about 40 min, or of about 45 min, or of about 50 min, or of about 60 min, or of about 70 min, or of about 80 min, or of about 90 min, or of about 100 mM. In one embodiment, the buffering agent is at a concentration of 1 min, or of 5 min, or of 10 min, or of 15 min, or of 20 min, or of 25 min, or of 30 min, or of 35 min, or of 40 min, or of 45 min, or of 50 min, or of 60 min, or of 70 min, or of 80 min, or of 90 min, or of 100 mM. In a specific embodiment, the buffering agent is at a concentration of between about 5 min and about 50 min. In another specific embodiment, the buffering agent is at a concentration of between 5 min and 20 min.

In certain embodiments, the formulation of the low impurity compositions, for example, low host cell protein compositions, of the invention comprises histidine as a buffering agent. In one embodiment the histidine is present in the formulation of the invention at a concentration of at least about 1 min, at least about 5 min, at least about 10 min, at least about 20 min, at least about 30 min, at least about 40 min, at least about 50 min, at least about 75 min, at least about 100 mM, at least about 150 min, or at least about 200 min histidine. In another embodiment, a formulation of the invention comprises between about 1 min and about 200 min, between about 1 min and about 150 min, between about 1 min and about 100 mM, between about 1 min and about 75 min, between about 10 min and about 200 min, between about 10 min and about 150 min, between about 10 min and about 100 mM, between about 10 min and about 75 min, between about 10 min and about 50 min, between about 10 min and about 40 min, between about 10 min and about 30 min, between about 20 min and about 75 min, between about 20 min and about 50 min, between about 20 min and about 40 min, or between about 20 min and about 30 min histidine. In a further embodiment, the formulation comprises about 1 min, about 5 min, about 10 min, about 20 min, about 25 min, about 30 min, about 35 min, about 40 min, about 45 min, about 50 min, about 60 min, about 70 min, about 80 min, about 90 min, about 100 mM, about 150 min, or about 200 min histidine. In a specific embodiment, a formulation may comprise about 10 min, about 25 min, or no histidine.

The formulations of the low impurity compositions, for example, low host cell protein compositions, of the invention may comprise a carbohydrate excipient. Carbohydrate excipients can act, e.g., as viscosity enhancing agents, stabilizers, bulking agents, solubilizing agents, and/or the like. Carbohydrate excipients are generally present at between about 1% to about 99% by weight or volume, e.g., between about 0.1% to about 20%, between about 0.1% to about 15%, between about 0.1% to about 5%, between about 1% to about 20%, between about 5% to about 15%, between about 8% to about 10%, between about 10% and about 15%, between about 15% and about 20%, between 0.1% to 20%, between 5% to 15%, between 8% to 10%, between 10% and 15%, between 15% and 20%, between about 0.1% to about 5%, between about 5% to about 10%, or between about 15% to about 20%. In still other specific embodiments, the carbohydrate excipient is present at 1%, or at 1.5%, or at 2%, or at 2.5%, or at 3%, or at 4%, or at 5%, or at 10%, or at 15%, or at 20%.

Carbohydrate excipients suitable for use in the formulations of the invention include, but are not limited to, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and the like. In one embodiment, the carbohydrate excipients for use in the present invention are chosen from, sucrose, trehalose, lactose, mannitol, and raffinose. In a specific embodiment, the carbohydrate excipient is trehalose. In another specific embodiment, the carbohydrate excipient is mannitol. In yet another specific embodiment, the carbohydrate excipient is sucrose. In still another specific embodiment, the carbohydrate excipient is raffinose. The purity of the carbohydrate excipient should be at least 98%, or at least 99%, or at least 99.5%.

In a specific embodiment, the formulations of the low impurity compositions, for example, low host cell compositions, of the invention may comprise trehalose. In one embodiment, a formulation of the invention comprises at least about 1%, at least about 2%, at least about 4%, at least about 8%, at least about 20%, at least about 30%, or at least about 40% trehalose. In another embodiment, a formulation of the invention comprises between about 1% and about 40%, between about 1% and about 30%, between about 1% and about 20%, between about 2% and about 40%, between about 2% and about 30%, between about 2% and about 20%, between about 4% and about 40%, between about 4% and about 30%, or between about 4% and about 20% trehalose. In a further embodiment, a formulation of the invention comprises about 1%, about 2%, about 4%, about 6%, about 8%, about 15%, about 20%, about 30%, or about 40% trehalose. In a specific embodiment, a formulation of the invention comprises about 4%, about 6% or about 15% trehalose.

In certain embodiments, a formulation of the low impurity compositions, for example, low host cell compositions, of the invention comprises an excipient. In a specific embodiment, a formulation of the invention comprises at least one excipient chosen from: sugar, salt, surfactant, amino acid, polyol, chelating agent, emulsifier and preservative. In one embodiment, a formulation of the invention comprises a salt, e.g., a salt selected from: NaCl, KCl, CaCl₂, and MgCl₂. In a specific embodiment, the formulation comprises NaCl.

A formulation of the low impurity compositions, for example, low host cell compositions, of the invention may comprise at least about 10 min, at least about 25 min, at least about 50 min, at least about 75 min, at least about 80 min, at least about 100 mM, at least about 125 min, at least about 150 min, at least about 175 min, at least about 200 min, or at least about 300 min sodium chloride (NaCl). In a further embodiment, the formulation may comprise between about 10 min and about 300 min, between about 10 min and about 200 min, between about 10 min and about 175 min, between about 10 min and about 150 min, between about 25 min and about 300 min, between about 25 min and about 200 min, between about 25 min and about 175 min, between about 25 min and about 150 min, between about 50 min and about 300 min, between about 50 min and about 200 min, between about 50 min and about 175 min, between about 50 min and about 150 min, between about 75 min and about 300 min, between about 75 min and about 200 min, between about 75 min and about 175 min, between about 75 min and about 150 min, between about 100 mM and about 300 min, between about 100 mM and about 200 min, between about 100 mM and about 175 min, or between about 100 mM and about 150 min sodium chloride. In a further embodiment, the formulation may comprise about 10 min, about 25 min, about 50 min, about 75 min, about 80 min, about 100 mM, about 125 min, about 150 min, about 175 min, about 200 min, or about 300 min sodium chloride.

A formulation of the low impurity compositions, for example, low host cell compositions, of the invention may also comprise an amino acid, e.g., lysine, arginine, glycine, histidine or an amino acid salt. The formulation may comprise at least about 1 min, at least about 10 min, at least about 25 min, at least about 50 min, at least about 100 mM, at least about 150 min, at least about 200 min, at least about 250 min, at least about 300 min, at least about 350 min, or at least about 400 min of an amino acid. In another embodiment, the formulation may comprise between about 1 min and about 100 mM, between about 10 min and about 150 min, between about 25 min and about 250 min, between about 25 min and about 300 min, between about 25 min and about 350 min, between about 25 min and about 400 min, between about 50 min and about 250 min, between about 50 min and about 300 min, between about 50 min and about 350 min, between about 50 min and about 400 min, between about 100 mM and about 250 min, between about 100 mM and about 300 min, between about 100 mM and about 400 min, between about 150 min and about 250 min, between about 150 min and about 300 min, or between about 150 min and about 400 min of an amino acid. In a further embodiment, a formulation of the invention comprises about 1 min, 1.6 min, 25 min, about 50 min, about 100 mM, about 150 min, about 200 min, about 250 min, about 300 min, about 350 min, or about 400 min of an amino acid.

The formulations of the low impurity compositions, for example, low host cell protein compositions, of the invention may further comprise a surfactant. The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials. Pharmaceutically acceptable surfactants like polysorbates (e.g., polysorbates 20 or 80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.), can optionally be added to the formulations of the invention to reduce aggregation. In one embodiment, a formulation of the invention comprises Polysorbate 20, Polysorbate 40, Polysorbate 60, or Polysorbate 80. Surfactants are particularly useful if a pump or plastic container is used to administer the formulation. The presence of a pharmaceutically acceptable surfactant mitigates the propensity for the protein to aggregate. The formulations may comprise a polysorbate which is at a concentration ranging from between about 0.001% to about 1%, or about 0.001% to about 0.1%, or about 0.01% to about 0.1%. In other specific embodiments, the formulations of the invention comprise a polysorbate which is at a concentration of 0.001%, or 0.002%, or 0.003%, or 0.004%, or 0.005%, or 0.006%, or 0.007%, or 0.008%, or 0.009%, or 0.01%, or 0.015%, or 0.02%.

The formulations of the low impurity compositions, for example, low host cell protein compositions, of the invention may optionally further comprise other common excipients and/or additives including, but not limited to, diluents, binders, stabilizers, lipophilic solvents, preservatives, adjuvants, or the like. Pharmaceutically acceptable excipients and/or additives may be used in the formulations of the invention. Commonly used excipients/additives, such as pharmaceutically acceptable chelators (for example, but not limited to, EDTA, DTPA or EGTA) can optionally be added to the formulations of the invention to reduce aggregation. These additives are particularly useful if a pump or plastic container is used to administer the formulation.

Preservatives, such as phenol, m-cresol, p-cresol, o-cresol, chlorocresol, benzyl alcohol, phenylmercuric nitrite, phenoxyethanol, formaldehyde, chlorobutanol, magnesium chloride (for example, but not limited to, hexahydrate), alkylparaben (methyl, ethyl, propyl, butyl and the like), benzalkonium chloride, benzethonium chloride, sodium dehydroacetate and thimerosal, or mixtures thereof can optionally be added to the formulations of the invention at any suitable concentration such as between about 0.001% to about 5%, or any range or value therein. The concentration of preservative used in the formulations of the invention is a concentration sufficient to yield a microbial effect. Such concentrations are dependent on the preservative selected and are readily determined by the skilled artisan.

Other contemplated excipients/additives, which may be utilized in the formulations of the invention include, for example, flavoring agents, antimicrobial agents, sweeteners, antioxidants, antistatic agents, lipids such as phospholipids or fatty acids, steroids such as cholesterol, protein excipients such as serum albumin (human serum albumin (HSA), recombinant human albumin (rHA), gelatin, casein, salt-forming counterions such as sodium and the like. These and additional known pharmaceutical excipients and/or additives suitable for use in the formulations of the invention are known in the art, e.g., as listed in “Remington: The Science & Practice of Pharmacy”, 21^{st} ed., Lippincott Williams & Wilkins, (2005), and in the “Physician's Desk Reference”, 60^{th} ed., Medical Economics, Montvale, N.J. (2005). Pharmaceutically acceptable carriers can be routinely selected that are suitable for the mode of administration, solubility and/or stability of antibody (e.g., a non-human antibody, such as a canine antibody), as well known those in the art or as described herein.

It will be understood by one skilled in the art that the formulations of the low impurity compositions, for example, low host cell protein compositions, of the invention may be isotonic with human blood, wherein the formulations of the invention have essentially the same osmotic pressure as human blood. Such isotonic formulations will generally have an osmotic pressure from about 250 mOSm to about 350 mOSm. Isotonicity can be measured by, for example, using a vapor pressure or ice-freezing type osmometer. Tonicity of a formulation is adjusted by the use of tonicity modifiers. “Tonicity modifiers” are those pharmaceutically acceptable inert substances that can be added to the formulation to provide an isotonicity of the formulation. Tonicity modifiers suitable for this invention include, but are not limited to, saccharides, salts and amino acids.

In certain embodiments, the formulations of the low impurity compositions, for example, low host cell compositions, of the invention have an osmotic pressure from about 100 mOSm to about 1200 mOSm, or from about 200 mOSm to about 1000 mOSm, or from about 200 mOSm to about 800 mOSm, or from about 200 mOSm to about 600 mOSm, or from about 250 mOSm to about 500 mOSm, or from about 250 mOSm to about 400 mOSm, or from about 250 mOSm to about 350 mOSm.

The concentration of any one component or any combination of various components, of the formulations of the low impurity compositions, for example, low host cell compositions, of the invention is adjusted to achieve the desired tonicity of the final formulation. For example, the ratio of the carbohydrate excipient to antibody (e.g., a non-human antibody, such as a canine antibody) may be adjusted according to methods known in the art (e.g., U.S. Pat. No. 6,685,940). In certain embodiments, the molar ratio of the carbohydrate excipient to antibody (e.g., a canine antibody) may be from about 100 moles to about 1000 moles of carbohydrate excipient to about 1 mole of antibody, or from about 200 moles to about 6000 moles of carbohydrate excipient to about 1 mole of antibody, or from about 100 moles to about 510 moles of carbohydrate excipient to about 1 mole of antibody, or from about 100 moles to about 600 moles of carbohydrate excipient to about 1 mole of antibody.

The desired isotonicity of the final formulation may also be achieved by adjusting the salt concentration of the formulations. Pharmaceutically acceptable salts and those suitable for this invention as tonicity modifiers include, but are not limited to, sodium chloride, sodium succinate, sodium sulfate, potassium chloride, magnesium chloride, magnesium sulfate, and calcium chloride. In specific embodiments, formulations of the invention comprise NaCl, MgCl₂, and/or CaCl₂. In one embodiment, concentration of NaCl is between about 75 min and about 150 min. In another embodiment, concentration of MgCl₂ is between about 1 min and about 100 mM. Pharmaceutically acceptable amino acids including those suitable for this invention as tonicity modifiers include, but are not limited to, proline, alanine, L-arginine, asparagine, L-aspartic acid, glycine, serine, lysine, and histidine.

In one embodiment the formulations of the low impurity compositions, for example, low host cell protein compositions, of the invention are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with antibodies of interest (e.g., a non-human antibody, such as a canine antibody), even trace amounts of harmful and dangerous endotoxin must be removed. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

When used for in vivo administration, the formulations of the low impurity compositions, for example, low host cell protein compositions, of the invention should be sterile. The formulations of the invention may be sterilized by various sterilization methods, including sterile filtration, radiation, etc. In one embodiment, the antibody (e.g., a non-human antibody, such as a canine antibody) formulation is filter-sterilized with a presterilized 0.22-micron filter. Sterile compositions for injection can be formulated according to conventional pharmaceutical practice as described in “Remington: The Science & Practice of Pharmacy”, 21^{st} ed., Lippincott Williams & Wilkins, (2005). Formulations comprising antibodies of interest (e.g., a canine antibody), such as those disclosed herein, ordinarily will be stored in lyophilized form or in solution. It is contemplated that sterile compositions comprising antibodies of interest (e.g., a canine antibody) are placed into a container having a sterile access port, for example, an intravenous solution bag or vial having an adapter that allows retrieval of the formulation, such as a stopper pierceable by a hypodermic injection needle. In one embodiment, a composition of the invention is provided as a pre-filled syringe.

In one embodiment, a formulation of the low impurity compositions, for example, low host cell protein compositions, of the invention is a lyophilized formulation. The term “lyophilized” or “freeze-dried” includes a state of a substance that has been subjected to a drying procedure such as lyophilization, where at least 50% of moisture has been removed.

The phrase “bulking agent” includes a compound that is pharmaceutically acceptable and that adds bulk to a lyo cake. Bulking agents known to the art include, for example, carbohydrates, including simple sugars such as dextrose, ribose, fructose and the like, alcohol sugars such as mannitol, inositol and sorbitol, disaccharides including trehalose, sucrose and lactose, naturally occurring polymers such as starch, dextrans, chitosan, hyaluronate, proteins (e.g., gelatin and serum albumin), glycogen, and synthetic monomers and polymers.

A “lyoprotectant” is a molecule which, when combined with an antibody (e.g., a non-human antibody, such as a canine antibody), significantly prevents or reduces chemical and/or physical instability of the protein upon lyophilization and subsequent storage. Lyoprotectants include, but are not limited to, sugars and their corresponding sugar alcohols; an amino acid such as monosodium glutamate or histidine; a methylamine such as betaine; a lyotropic salt such as magnesium sulfate; a polyol such as trihydric or higher molecular weight sugar alcohols, e.g., glycerin, dextran, erythritol, glycerol, arabitol, xylitol, sorbitol, and mannitol; propylene glycol; polyethylene glycol; PLURONICS™; and combinations thereof. Additional examples of lyoprotectants include, but are not limited to, glycerin and gelatin, and the sugars mellibiose, melezitose, raffinose, mannotriose and stachyose. Examples of reducing sugars include, but are not limited to, glucose, maltose, lactose, maltulose, iso-maltulose and lactulose. Examples of non-reducing sugars include, but are not limited to, non-reducing glycosides of polyhydroxy compounds selected from sugar alcohols and other straight chain polyalcohols. Examples of sugar alcohols include, but are not limited to, monoglycosides, compounds obtained by reduction of disaccharides such as lactose, maltose, lactulose and maltulose. The glycosidic side group can be either glucosidic or galactosidic. Additional examples of sugar alcohols include, but are not limited to, glucitol, maltitol, lactitol and iso-maltulose. In specific embodiments, trehalose or sucrose is used as a lyoprotectant.

The lyoprotectant is added to the pre-lyophilized formulation in a “lyoprotecting amount” which means that, following lyophilization of the protein in the presence of the lyoprotecting amount of the lyoprotectant, the antibody essentially retains its physical and chemical stability and integrity upon lyophilization and storage.

In one embodiment, the molar ratio of a lyoprotectant (e.g., trehalose) and antibody (e.g., a non-human antibody, such as a canine antibody) molecules of a formulation of the invention is at least about 10, at least about 50, at least about 100, at least about 200, or at least about 300. In another embodiment, the molar ratio of a lyoprotectant (e.g., trehalose) and antibody molecules of a formulation of the invention is about 1, is about 2, is about 5, is about 10, about 50, about 100, about 200, or about 300.

A “reconstituted” formulation is one which has been prepared by dissolving a lyophilized antibody (e.g., a non-human antibody, such as a canine antibody) formulation in a diluent such that the antibody is dispersed in the reconstituted formulation. The reconstituted formulation is suitable for administration (e.g., parenteral administration) to a patient to be treated with the antibody and, in certain embodiments of the invention, may be one which is suitable for intravenous administration.

The “diluent” of interest herein is one which is pharmaceutically acceptable (safe and non-toxic for administration to a human) and is useful for the preparation of a liquid formulation, such as a formulation reconstituted after lyophilization. In some embodiments, diluents include, but are not limited to, sterile water, bacteriostatic water for injection (BWFI), a pH buffered solution (e.g., phosphate-buffered saline), sterile saline solution, Ringer's solution or dextrose solution. In an alternative embodiment, diluents can include aqueous solutions of salts and/or buffers.

In certain embodiments, a formulation of the low impurity compositions, for example, low host cell protein compositions, of the invention is a lyophilized formulation comprising an antibody (e.g., a non-human antibody, such as a canine antibody) of the invention, wherein at least about 90%, at least about 95%, at least about 97%, at least about 98%, or at least about 99% of said antibody may be recovered from a vial upon shaking said vial for 4 hours at a speed of 400 shakes per minute wherein the vial is filled to half of its volume with the formulation. In another embodiment, a formulation of the invention is a lyophilized formulation comprising an antibody of the invention, wherein at least about 90%, at least about 95%, at least about 97%, at least about 98%, or at least about 99% of the antibody may be recovered from a vial upon subjecting the formulation to three freeze/thaw cycles wherein the vial is filled to half of its volume with said formulation. In a further embodiment, a formulation of the invention is a lyophilized formulation comprising an antibody of the invention, wherein at least about 90%, at least about 95%, at least about 97%, at least about 98%, or at least about 99% of the antibody may be recovered by reconstituting a lyophilized cake generated from said formulation.

In one embodiment, a reconstituted liquid formulation may comprise an antibody (e.g., a non-human antibody, such as a canine antibody) at the same concentration as the pre-lyophilized liquid formulation.

In another embodiment, a reconstituted liquid formulation may comprise an antibody (e.g., a non-human antibody, such as a canine antibody) at a higher concentration than the pre-lyophilized liquid formulation, e.g., about 2 fold, about 3 fold, about 4 fold, about 5 fold, about 6 fold, about 7 fold, about 8 fold, about 9 fold, or about 10 fold higher concentration of an antibody than the pre-lyophilized liquid formulation.

In yet another embodiment, a reconstituted liquid formulation may comprise an antibody (e.g., a non-human antibody, such as a canine antibody) of the invention at a lower concentration than the pre-lyophilized liquid formulation, e.g., about 2 fold, about 3 fold, about 4 fold, about 5 fold, about 6 fold, about 7 fold, about 8 fold, about 9 fold or about 10 fold lower concentration of an antibody than the pre-lyophilized liquid formulation.

The pharmaceutical formulations of the low impurity compositions, for example, low host cell protein compositions, of the invention are typically stable formulations, e.g., stable at room temperature.

The terms “stability” and “stable” as used herein in the context of a formulation comprising an antibody (e.g., a non-human antibody, such as a canine antibody) of the invention refer to the resistance of the antibody in the formulation to aggregation, degradation or fragmentation under given manufacture, preparation, transportation and storage conditions. The “stable” formulations of the invention retain biological activity under given manufacture, preparation, transportation and storage conditions. The stability of the antibody can be assessed by degrees of aggregation, degradation or fragmentation, as measured by HPSEC, static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation. For example, a reference formulation may be a reference standard frozen at −70° C. consisting of 10 mg/ml of an antibody of the invention in PBS.

Therapeutic formulations of the low impurity compositions, for example, low host cell protein compositions, of the invention may be formulated for a particular dosage. Dosage regimens may be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the antibody (e.g., a non-human antibody, such as a canine antibody) and the particular therapeutic effect to be achieved, and (b) the limitations inherent in the art of compounding such an antibody for the treatment of sensitivity in individuals.

Therapeutic compositions of the low impurity compositions, for example, low host cell compositions, of the invention can be formulated for particular routes of administration, such as oral, nasal, pulmonary, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. By way of example, in certain embodiments, the antibodies of interest (including fragments of the antibody) are formulated for intravenous administration. In certain other embodiments, the antibody (e.g., a non-human antibody, such as a canine antibody), including fragments of the antibody are formulated for local delivery to the cardiovascular system, for example, via catheter, stent, wire, intramyocardial delivery, intrapericardial delivery, or intraendocardial delivery.

Formulations of the low impurity compositions, for example, low host cell protein compositions, of the invention which are suitable for topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required (U.S. Pat. Nos. 7,378,110; 7,258,873; 7,135,180; 7,923,029; and US Publication No. 20040042972).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Actual dosage levels of the active ingredients in the pharmaceutical compositions of the low impurity compositions, for example, low host cell compositions, of the invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

In certain embodiments, the antibodies of interest of the invention can be formulated to ensure proper distribution in vivo. For example, the blood-brain bather (BBB) excludes many highly hydrophilic compounds. To ensure that the therapeutic compounds of the invention can cross the BBB (if desired), they can be formulated, for example, in liposomes. For methods of manufacturing liposomes, see, e.g., U.S. Pat. Nos. 4,522,811; 5,374,548; 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant Protein A receptor (Briscoe et al. (1995) Am. J. Physiol. 1233:134), different species of which may comprise the formulations of the invention, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In one embodiment of the invention, the therapeutic compounds of the invention are formulated in liposomes; in another embodiment, the liposomes include a targeting moiety. In another embodiment, the therapeutic compounds in the liposomes are delivered by bolus injection to a site proximal to the desired area. When administered in this manner, the composition must be fluid to the extent that easy syringability exists. It must be stable under the conditions of manufacture and storage and may be preserved against the contaminating action of microorganisms such as bacteria and fungi. Additionally or alternatively, the antibodies of interest (e.g., a non-human antibody, such as a canine antibody) of the invention may be delivered locally to the brain to mitigate the risk that the blood brain barrier slows effective delivery.

In certain embodiments, the low impurity compositions, for example, low host cell protein compositions, of the invention may be administered with medical devices known in the art. For example, in certain embodiments an antibody (e.g., a non-human antibody, such as a canine antibody) or a fragment of the antibody is administered locally via a catheter, stent, wire, or the like. For example, in one embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

The efficient dosages and the dosage regimens for the reduced level of at least one impurity compositions of the invention depend on the disease or condition to be treated and can be determined by the persons skilled in the art. One of ordinary skill in the art would be able to determine such amounts based on such factors as the subject's size, the severity of the subject's symptoms, and the particular composition or route of administration selected.

### Alternative Formulations Containing the Low Impurity Compositions of the Invention

### Alternative Aqueous Formulations

The invention also provides a low impurity composition, for example a low host cell protein composition, formulated as an aqueous formulation comprising an antibody and water, as described in U.S. Pat. No. 8,420,081, the contents of which are hereby incorporated by reference. In these aqueous formulations, the antibody is stable without the need for additional agents. This aqueous formulation has a number of advantages over conventional formulations in the art, including stability of the antibody in water without the requirement for additional excipients, increased concentrations of the antibody without the need for additional excipients to maintain solubility of the antibody, and low osmolality. These also have advantageous storage properties, as the antibodies of interest in the formulation remain stable during storage, e.g., stored as a liquid form for more than 3 months at 7° C. or freeze/thaw conditions, even at high antibody concentrations and repeated freeze/thaw processing steps. In one embodiment, formulations described herein include high concentrations of antibodies of interest such that the aqueous formulation does not show significant opalescence, aggregation, or precipitation.

In one embodiment, an aqueous low impurity composition comprising an antibody, e.g., a non-human antibody, such as a canine antibody and water is provided, wherein the formulation has certain characteristics, such as, but not limited to, low conductivity, e.g., a conductivity of less than about 2.5 mS/cm, an antibody concentration of at least about 10 μg/mL, an osmolality of no more than about 30 mOsmol/kg, and/or the antibody has a molecular weight (Mw) greater than about 47 kDa. In one embodiment, the formulation has improved stability, such as, but not limited to, stability in a liquid form for an extended time (e.g., at least about 3 months or at least about 12 months) or stability through at least one freeze/thaw cycle (if not more freeze/thaw cycles). In one embodiment, the formulation is stable for at least about 3 months in a form selected from the group consisting of frozen, lyophilized, or spray-dried.

In one embodiment, the formulation has a low conductivity, including, for example, a conductivity of less than about 2.5 mS/cm, a conductivity of less than about 2 mS/cm, a conductivity of less than about 1.5 mS/cm, a conductivity of less than about 1 mS/cm, or a conductivity of less than about 0.5 mS/cm.

In another embodiment, low impurity compositions included in the formulation have a given concentration, including, for example, a concentration of at least about 1 mg/mL, at least about 10 mg/mL, at least about 50 mg/mL, at least about 100 mg/mL, at least about 150 mg/mL, at least about 200 mg/mL, or greater than about 200 mg/mL. In another embodiment, the formulation of the invention has an osmolality of no more than about 15 mOsmol/kg.

The aqueous formulations described herein do not rely on standard excipients, e.g., a tonicity modifier, a stabilizing agent, a surfactant, an anti-oxidant, a cryoprotectant, a bulking agent, a lyroprotectant, a basic component, and an acidic component. In other embodiments of the invention, the formulation contains water, one or more antibody, and no ionic excipients (e.g., salts, free amino acids).

In certain embodiments, the aqueous formulation as described herein comprise a low impurity composition comprising an antibody concentration of at least 50 mg/mL and water, wherein the formulation has an osmolality of no more than 30 mOsmol/kg. Lower limits of osmolality of the aqueous formulation are also encompassed by the invention. In one embodiment the osmolality of the aqueous formulation is no more than 15 mOsmol/kg. The aqueous formulation of the invention may have an osmolality of less than 30 mOsmol/kg, and also have a high antibody concentration, e.g., the concentration of the antibody is at least 100 mg/mL, and may be as much as 200 mg/mL or greater. Ranges intermediate to the above recited concentrations and osmolality units are also intended to be part of this invention. In addition, ranges of values using a combination of any of the above recited values as upper and/or lower limits are intended to be included.

The concentration of the aqueous formulation as described herein is not limited by the antibody size and the formulation may include any size range of antibodies. Included within the scope of the invention is an aqueous formulation comprising at least 40 mg/mL and as much as 200 mg/mL or more of an antibody, for example, 40 mg/mL, 65 mg/mL, 130 mg/mL, or 195 mg/ml, which may range in size from 5 kDa to 150 kDa or more. In one embodiment, the antibody in the formulation of the invention is at least about 15 kD in size, at least about 20 kD in size; at least about 47 kD in size; at least about 60 kD in size; at least about 80 kD in size; at least about 100 kD in size; at least about 120 kD in size; at least about 140 kD in size; at least about 160 kD in size; or greater than about 160 kD in size. Ranges intermediate to the above recited sizes are also intended to be part of this invention. In addition, ranges of values using a combination of any of the above recited values as upper and/or lower limits are intended to be included.

The aqueous formulation as described herein may be characterized by the hydrodynamic diameter (Dₕ) of the antibodies of interest in solution. The hydrodynamic diameter of the antibody in solution may be measured using dynamic light scattering (DLS), which is an established analytical method for determining the Dₕ of proteins. Typical values for monoclonal antibodies, e.g., IgG, are about 10 nm Low-ionic formulations may be characterized in that the Dₕ of the antibodies of interest are notably lower than antibody formulations comprising ionic excipients. It has been discovered that the Dₕ values of antibodies in aqueous formulations made using the disfiltration/ultrafilteration (DF/UF) process, as described in U.S. Pat. No. 8,420,081, using pure water as an exchange medium, are notably lower than the Dₕ of antibodies in conventional formulations independent of protein concentration. In one embodiment, antibodies in the aqueous formulation as described herein have a Dₕ of less than 4 nm, or less than 3 nm.

In one embodiment, the Dₕ of the antibody in the aqueous formulation is smaller relative to the Dₕ of the same antibody in a buffered solution, irrespective of antibody concentration. Thus, in certain embodiments, a antibody in an aqueous formulation made in accordance with the methods described herein, will have a Dₕ which is at least 25% less than the Dₕ of the antibody in a buffered solution at the same given concentration. Examples of buffered solutions include, but are not limited to phosphate buffered saline (PBS). In certain embodiments, antibodies of interest in the aqueous formulation of the invention have a Dₕ that is at least 50% less than the Dₕ of the antibody in PBS in at the given concentration; at least 60% less than the Dₕ of the antibody in PBS at the given concentration; at least 70% less than the Dₕ of the antibody in PBS at the given concentration; or more than 70% less than the Dₕ of the antibody in PBS at the given concentration. Ranges intermediate to the above recited percentages are also intended to be part of this invention, e.g., about 55%, 56%, 57%, 64%, 68%, and so forth. In addition, ranges of values using a combination of any of the above recited values as upper and/or lower limits are intended to be included, e.g., about 50% to about 80%.

In one aspect, the aqueous formulation includes the antibody at a dosage of about 0.01 mg/kg-10 mg/kg. In another aspect, the dosages of the antibody include approximately 1 mg/kg administered every other week, or approximately 0.3 mg/kg administered weekly. A skilled practitioner can ascertain the proper dosage and regime for administering to a subject.

### Alternative Solid Unit Formulations

The invention also provides a low impurity composition of the invention formulated as a stable composition of a antibody, e.g., an antibody, or antigen binding portion thereof, and a stabilizer, referred to herein as solid units, as described in U.S. Provisional Application No. 61/893,123, the contents of which are hereby incorporated by reference herein.

Specifically, it has been discovered that despite having a high proportion of sugar, the solid units comprising the low impurity compositions of the invention maintain structural rigidity and resist changes in shape and/or volume when stored under ambient conditions, e.g., room temperature and humidity, for extended periods of time (e.g., the solid units comprising the low impurity compositions of the invention do not require storage in a sealed container) and maintain long-term physical and chemical stability of the antibody without significant degradation and/or aggregate formation. Moreover, despite having a high proportion of sugar, the solid units comprising the low impurity compositions of the invention remain free-flowing when stored under ambient conditions, e.g., room temperature and humidity, for extended periods of time, and yet are easily dissolved in an aqueous solvent, e.g., water (e.g., the solid units require minimal mixing when contacted with a solvent for reconstitution). Furthermore, the solid units comprising the low impurity compositions of the invention may be prepared directly in a device for patient use. These properties, when compared to existing techniques which require a vial containing a lyophilized antibody provided as a cake (which may not stabilize a antibody for extended periods of time), a separate vial for a diluent, one or more sterile syringes, and several manipulation steps, thus provides alternative approaches for reconstitution since the solid units comprising the low impurity compositions of the invention may be provided, e.g., in a dual chambered cartridge, to make reconstitution invisible during patient delivery. Furthermore, the solid units comprising the low impurity compositions of the invention are versatile in that they can be readily and easily adapted for numerous modes of administration, such as parenteral and oral administration.

As used herein, the term “solid unit,” refers to a composition which is suitable for pharmaceutical administration and comprises a antibody, e.g., an antibody or peptide, and a stabilizer, e.g., a sugar. The solid unit comprising the low impurity compositions of the invention has a structural rigidity and resistance to changes in shape and/or volume. In one embodiment, the solid unit comprising the low impurity compositions of the invention is obtained by freeze-drying a pharmaceutical formulation of a therapeutic antibody. The solid unit comprising the low impurity compositions of the invention may be any shape, e.g., geometric shape, including, but not limited to, a sphere, a cube, a pyramid, a hemisphere, a cylinder, a teardrop, and so forth, including irregularly shaped units. In one embodiment, the solid unit has a volume ranging from about 1 μl to about 20 μl. In another embodiment, the solid unit is not obtained using spray drying techniques, e.g., the solid unit is not a powder or granule.

As used herein, the phrase “a plurality of solid units” refers to a collection or population of solid units comprising the low impurity compositions of the invention, wherein the collection comprises two or more solid units having a substantially uniform shape, e.g., sphere, and/or volume distribution. A substantially uniform size distribution is intended to mean that the individual shapes and/or volumes of the solid units comprising the low impurity compositions of the invention are substantially similar and not greater than a 10% standard deviation in volume. For example, a plurality of solid units which are spherical in shape would include a collection of solid units having no greater than 10% standard deviation from an average volume of the spheres. In one embodiment, the plurality of solid units is free-flowing.

### Kits and Articles of Manufacture Comprising the Low Impurity Compositions of the Invention

Also within the scope of the present invention are kits comprising the low impurity compositions of the invention and instructions for use. The term “kit” as used herein refers to a packaged product comprising components with which to administer the antibody (e.g., a non-human antibody, such as a canine antibody), of the invention for treatment of a disease or disorder. The kit may comprise a box or container that holds the components of the kit. The box or container is affixed with a label or a Food and Drug Administration approved protocol. The box or container holds components of the invention which may be contained within plastic, polyethylene, polypropylene, ethylene, or propylene vessels. The vessels can be capped-tubes or bottles. The kit can also include instructions for administering a antibody (e.g., a canine antibody) of the invention.

The kit can further contain one more additional reagents, such as an immunosuppressive reagent, a cytotoxic agent or a radiotoxic agent or one or more additional antibodies of interest of the invention (e.g., aa non-human antibody, such as a canine antibody). Kits typically include a label indicating the intended use of the contents of the kit. The term label includes any writing, or recorded material supplied on or with the kit, or which otherwise accompanies the kit.

The invention also provides a pharmaceutical pack or kit comprising one or more containers filled with a liquid formulation or lyophilized formulation of an antibody (e.g., a non-human antibody, such as a canine antibody) of the invention. In one embodiment, a container filled with a liquid formulation of the invention is a pre-filled syringe. In a specific embodiment, the formulations of the invention are formulated in single dose vials as a sterile liquid. For example, the formulations may be supplied in 3 cc USP Type I borosilicate amber vials (West Pharmaceutical Services—Part No. 6800-0675) with a target volume of 1.2 mL. Optionally associated with such container(s) can be a notice in the form prescribed by a governmental agency regulating the manufacture, use or sale of pharmaceuticals or biological products, which notice reflects approval by the agency of manufacture, use or sale for human administration.

In one embodiment, a container filled with a liquid formulation of the invention is a pre-filled syringe. Any pre-filled syringe known to one of skill in the art may be used in combination with a liquid formulation of the invention. Pre-filled syringes that may be used are described in, for example, but not limited to, PCT Publications WO05032627, WO08094984, WO9945985, WO03077976, U.S. Pat. No. 6,792,743, U.S. Pat. No. 5,607,400, U.S. Pat. No. 5,893,842, U.S. Pat. No. 7,081,107, U.S. Pat. No. 7,041,087, U.S. Pat. No. 5,989,227, U.S. Pat. No. 6,807,797, U.S. Pat. No. 6,142,976, U.S. Pat. No. 5,899,889, U.S. Pat. No. 7,699,811, U.S. Pat. No. 7,540,382, U.S. Pat. No. 7,998,120, U.S. Pat. No. 7,645,267, and US Patent Publication No. US20050075611. Pre-filled syringes may be made of various materials. In one embodiment a pre-filled syringe is a glass syringe. In another embodiment a pre-filled syringe is a plastic syringe. One of skill in the art understands that the nature and/or quality of the materials used for manufacturing the syringe may influence the stability of a protein formulation stored in the syringe. For example, it is understood that silicon based lubricants deposited on the inside surface of the syringe chamber may affect particle formation in the protein formulation. In one embodiment, a pre-filled syringe comprises a silicone based lubricant. In one embodiment, a pre-filled syringe comprises baked on silicone. In another embodiment, a pre-filled syringe is free from silicone based lubricants. One of skill in the art also understands that small amounts of contaminating elements leaching into the formulation from the syringe barrel, syringe tip cap, plunger or stopper may also influence stability of the formulation. For example, it is understood that tungsten introduced during the manufacturing process may adversely affect formulation stability. In one embodiment, a pre-filled syringe may comprise tungsten at a level above 500 ppb. In another embodiment, a pre-filled syringe is a low tungsten syringe. In another embodiment, a pre-filled syringe may comprise tungsten at a level between about 500 ppb and about 10 ppb, between about 400 ppb and about 10 ppb, between about 300 ppb and about 10 ppb, between about 200 ppb and about 10 ppb, between about 100 ppb and about 10 ppb, between about 50 ppb and about 10 ppb, between about 25 ppb and about 10 ppb.

In certain embodiments, kits comprising antibodies of interest (e.g., antibodies) of the invention are also provided that are useful for various purposes, e.g., research and diagnostic including for purification or immunoprecipitation of antibody from cells, detection of the antibody in vitro or in vivo. For isolation and purification of a antibody, the kit may contain an antibody coupled to beads (e.g., sepharose beads). Kits may be provided which contain the antibodies for detection and quantitation of a antibody in vitro, e.g., in an ELISA or a Western blot. As with the article of manufacture, the kit comprises a container and a label or package insert on or associated with the container. The container holds a composition comprising at least one antibody (e.g., a non-human antibody, such as a canine antibody) of the invention. Additional containers may be included that contain, e.g., diluents and buffers, control antibodies of interest (e.g., a canine antibody). The label or package insert may provide a description of the composition as well as instructions for the intended in vitro or diagnostic use.

The present invention also encompasses a finished packaged and labeled pharmaceutical product. This article of manufacture includes the appropriate unit dosage form in an appropriate vessel or container such as a glass vial, pre-filled syringe or other container that is hermetically sealed. In one embodiment, the unit dosage form is provided as a sterile particulate free solution comprising an antibody (e.g., a non-human antibody, such as a canine antibody) that is suitable for parenteral administration. In another embodiment, the unit dosage form is provided as a sterile lyophilized powder comprising an antibody (e.g., a canine antibody) that is suitable for reconstitution.

In one embodiment, the unit dosage form is suitable for intravenous, intramuscular, intranasal, oral, topical or subcutaneous delivery. Thus, the invention encompasses sterile solutions suitable for each delivery route. The invention further encompasses sterile lyophilized powders that are suitable for reconstitution.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question, as well as how and how frequently to administer the pharmaceutical. In other words, the article of manufacture includes instruction means indicating or suggesting a dosing regimen including, but not limited to, actual doses, monitoring procedures, and other monitoring information.

Specifically, the invention provides an article of manufacture comprising packaging material, such as a box, bottle, tube, vial, container, pre-filled syringe, sprayer, insufflator, intravenous (i.v.) bag, envelope and the like; and at least one unit dosage form of a pharmaceutical agent contained within said packaging material, wherein said pharmaceutical agent comprises a liquid formulation containing an antibody (e.g., a non-human antibody, such as a canine antibody). The packaging material includes instruction means which indicate how that said antibody (e.g., a canine antibody) can be used to prevent, treat and/or manage one or more symptoms associated with a disease or disorder

The present invention is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are expressly incorporated herein by reference. The entire contents of the following applications are also expressly incorporated herein by reference:

U.S. Provisional Patent Application 61/893,123, entitled “STABLE SOLID PROTEIN COMPOSITIONS AND METHODS OF MAKING SAME”, Attorney Docket Number 117813-31001, filed on Oct. 18, 2013;

U.S. Provisional Application Ser. No. 61/892,833, entitled “LOW ACIDIC SPECIES COMPOSITIONS AND METHODS FOR PRODUCING THE SAME USING DISPLACEMENT CHROMATOGRAPHY”, Attorney Docket Number 117813-73602, filed on Oct. 18, 2013;

U.S. Provisional Patent Application 61/892,710, entitled “MUTATED ANTI-TNFa ANTIBODIES AND METHODS OF THEIR USE”, Attorney Docket Number 117813-73802, filed on Oct. 18, 2013;

U.S. Provisional Patent Application 61/893,068, entitled “LOW ACIDIC SPECIES COMPOSITIONS AND METHODS FOR PRODUCING THE SAME”, Attorney Docket Number 117813-73901, filed on Oct. 18, 2013;

U.S. Provisional Patent Application 61/893,088, entitled “MODULATED LYSINE VARIANT SPECIES AND METHODS FOR PRODUCING AND USING THE SAME”, Attorney Docket Number 117813-74101, filed on Oct. 18, 2013; and

U.S. Provisional Patent Application 61/893,131, entitled “PURIFICATION OF PROTEINS USING HYDROPHOBIC INTERACTION CHROMATOGRAPHY”, Attorney Docket Number 117813-74301, filed on Oct. 18, 2013.

### EXAMPLES

### Examples 1

### Effect of MAb Concentration and Kosmotropic Salts on Static Binding Capacity of MabSelect SuRe Protein A Resin for Canine MAb A

The static binding capacity (Qs) of MabSelect SuRe Protein A resin for a Canine MAb A was measured at various feed concentration and salt conditions. In one experiment, a semi-purified canine MAb feed was used to evaluate the Qs values for the resin at different protein concentration. 500 ul of 20% MabSelect SuRe resin slurry was first transferred into a 7 mL size filter column. The resin was washed with 2 mL of water, followed by 2 mL of 0.1 M acetic acid pH 3.5 solution, 4 mL of water and then 5 mL of equilibration buffer which consisted of 50 mM Tris, 100 mM NaCl at pH 7.0. The canine MAb A feed was conditioned to ˜pH 7.1 and conductivity ˜11.6 mS/cm with final concentration ranging from 0.9 to 4.5 g/L. The resin was incubated with 1.9 to 4.5 mL of each feed on a rotating mixed for 2 hours at room temperature. After adsorption, the resin-protein slurries were filtered and the filtrates were collected. The resins were then washed with 2 mL of equilibration buffer followed by incubation with 2 mL of 20 mM Tris, pH 8.5, 0.6 mS/cm elution buffer for 30 min. The resin slurries were filtered again and filtrate collected into clean tube. The resin was then rinsed with 1 mL of elution buffer and the filtrate was collected and combined with the first eluate sample. These eluate samples were then measured by UV280 and Poros G HPLC assays to determine the canine MAb concentration. The Qs values were calculated based on the measured concentrations.

In another set of experiment, 500 ul of 20% MabSelect SuRe resin slurry was first transferred into a 7 mL size filter column. The resin was washed with 2 mL of water, followed by 2 mL of 0.1 M acetic acid pH 3.5 buffer, 4 mL of water and then 5 mL of various equilibration buffer. The equilibration buffer consisted of 40 mM Tris at pH 7.5 and 0.3 to 1.1 M (NH₄)₂SO₄, or 0.3 to 0.6 M Na₂SO₄, or 0.3 to 0.6 M NaCitrate, or none of these salts. The resin was equilibrated with each equilibration buffer before contact with a clarified canine MAb A harvest, which was supplemented with the various salts at concentrations identical to those of the equilibration buffer. The protein concentrations in the conditioned feed samples were between 3.2 to 4.7 g/L. The resin was incubated with 2.25 mL of each feed on a rotating mixed for 2 hours at room temperature. After adsorption, the resin-protein slurries were filtered and the filtrates were collected. The resins were then washed with 2 mL of equilibration buffer followed by incubation with 2 mL of 20 mM Tris, pH 8.5, 0.6 mS/cm elution buffer for 30 min. The resin slurries were filtered again and filtrate collected into clean tube. The resin was then rinsed with 1 mL of elution buffer and the filtrate was collected and combined with the first eluate sample. These eluate samples were then measured by Poros G HPLC assays to determine the canine MAb concentration. The Qs values were calculated based on the measured concentrations.

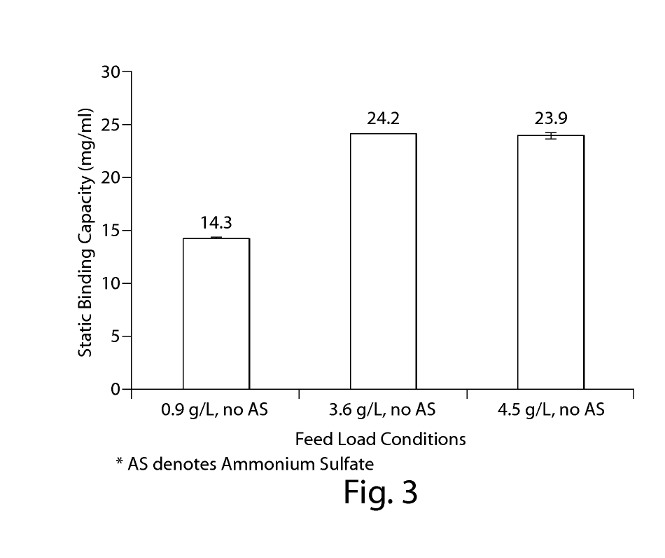

Unlike typical human antibodies, the canine MAb A has significantly lower binding capacity for Protein A, thus its static binding capacity on a standard commercial Protein A resin such as MabSelect SuRe is substantially lower. As shown in FIG. 3, the concentration of this MAb A in the load can significantly affect its Qs on the MabSelect SuRe resin. Increasing MAb A concentration from 0.9 g/L to 4.5 g/L increased the Qs from about 14 g/L to about 24 g/L, although changing the load concentration of 3.6 to 4.5 g/L did not affect Qs value. Thus, pre-concentrating a low titer (e.g. <1 g/L) clarified harvest of canine MAb A should enhance the Protein A binding capacity and throughput during its capture process.

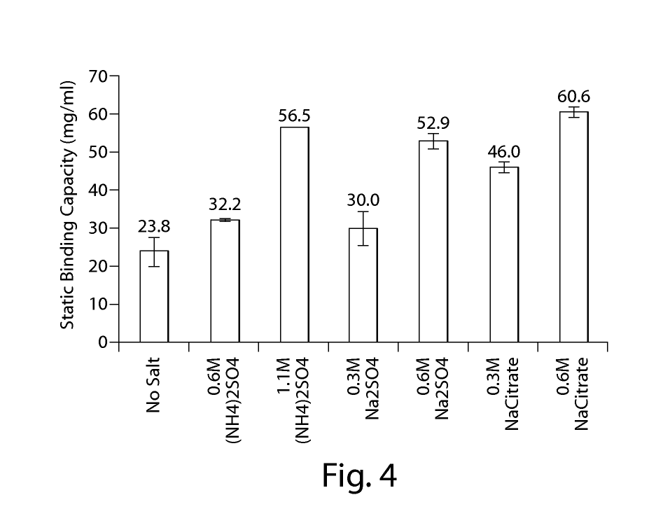

FIG. 4 shows the effects of various kosmotropic salts and their concentrations on the Qs of MabSelect SuRe Protein A resin for canine MAb A. Clearly, adding the kosmotropic salt such as (NH₄)₂SO₄, Na₂SO₄, or NaCitrate increases the Qs values dramatically; and the higher the salt concentration the higher the Qs. In the absence of the salt, the MabSelect SuRe resin gives ˜24 g/L total binding capacity at a feed MAb concentration of 4.7 g/L. In the presence of 1.1 M (NH₄)₂SO₄, the Qs increases to −57 g/L at a feed MAb concentration of 4.0 g/L. The latter Qs value reflects a typically observed static binding capacity for a standard, high affinity antibody on the MabSelect SuRe resin (i.e. 50-60 g/L). Consistent with “Hofmeister” series, NaCitrate is the most effective among the three salts in terms of boosting up the Qs at a given salt concentration. The Na₂SO₄ is also more effective than (NH₄)₂SO₄, and it increases Qs to −53 g/L at concentration of 0.6 M versus ˜32 g/L for the same concentration of (NH₄)₂SO₄, Nevertheless, all these salts can be used to effectively enhance the canine MAb A static binding capacity on a Protein A resin.

### Example 2

### Effect of MAb Concentration and Ammonium Sulfate on Dynamic Binding Capacity of Canine MAb A on MabSelect Sure Protein A Resin

The dynamic binding capacity (DBC) of canine MAb A on a MabSelect SuRe Protein A column was first measured using a clarified harvest in the absence of (NH₄)₂SO₄ or other kosmotropic salt. A canine MAb A clarified harvest (initially at ˜1.0 g/L titer) was first concentrated by 8-fold using a 30 kD Biomax membrane cassette. The concentrated harvest was 0.22 um filtered and then diluted with phosphate-buffered saline (PBS) solution to obtain final protein concentration of 0.8-5.6 g/L. These conditioned harvest feeds were used as the load material for MabSelect SuRe column. The column was first equilibrated with PBS buffer followed by feed loading at a flow rate corresponding to 4 min residence time (RT). The flow-through fractions were collected and measured using a Poros G assay to quantify MAb A concentrations which were used to determine the breakthrough curves. After feed loading, the MabSelect SuRe column was washed with equilibration buffer and then eluted with 20 mM Tris, pH 8.5 buffer (This MAb is not stable at low pH so standard low pH elution cannot be used here). The column was then regenerated with 0.15 M phosphoric acid followed by 0.1 M NaOH cleaning before next use.

The DBCs for canine MAb A was also measured in the presence of 1 M (NH₄)₂SO₄. Again, the original canine MAb A clarified harvest (at ˜1.0 g/L titer) was first concentrated by 8-fold using a 30 kD Biomax membrane cassette. The concentrated harvest was diluted with 40 mM Tris, 2.2 M (NH₄)₂SO₄, pH 7.5 solution to obtain final protein concentration of 5.3 g/L and (NH₄)₂SO₄ concentration of 1 M. This material was then 0.22 um filtered to remove haziness. There was no product loss during these preparation steps. The concentrated harvest feed was used to determine the DBC of the MabSelect SuRe resin with 1 M (NH₄)₂SO₄ in the feed and 1.1 M (NH₄)₂SO₄ in the EQ/wash buffer. The DBC run was carried out on MabSelect SuRe column at 4 min and 6 min RT flow rates. In another run, the concentrated feed was also diluted to ˜3 g/L and then diluted with 2.2 M (NH₄)₂SO₄ to obtain 1 M (NH₄)₂SO₄ and final MAb concentration of 1.7 g/L, and the DBC of MabSelect SuRe resin at 6 min RT was determined with this material. The flow-through fractions during each run were collected and analyzed by Poros G assay to determine the breakthrough curve. The column elution and regeneration were identical to those described above.

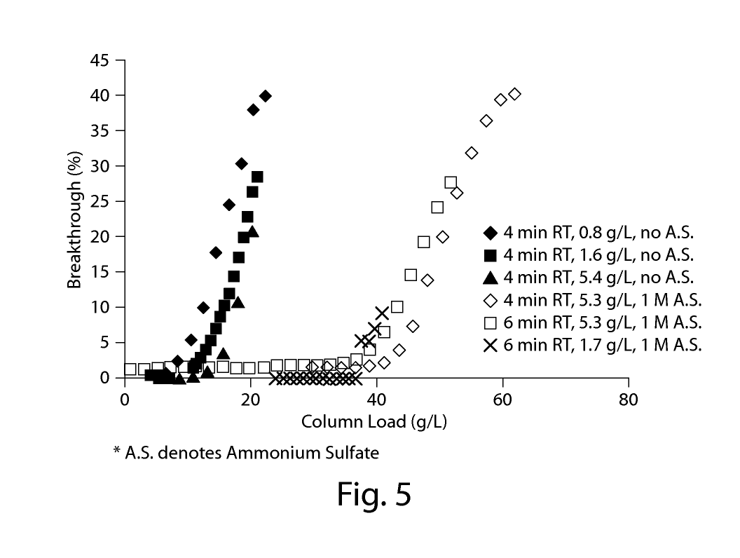

FIG. 5 shows the breakthrough curves for canine MAb A on MabSelect SuRe Protein A column in the absence and presence of (NH₄)₂SO₄ and at various MAb concentration and RT. When there was no (NH₄)₂SO₄ in the load sample, the protein breakthrough occurred much earlier (i.e. <20 g/L resin load), and increasing MAb concentration in the load delayed the breakthrough, consistent with Qs data shown in Example 1. In comparison, adding 1 M (NH₄)₂SO₄ in the load is much more effective in increasing DBCs as the breakthrough curves shifted to much higher column loading level. The breakthrough curves were not significantly affected by the MAb concentration in the range of 1.7 to 5.3 g/L or the flow residence time from 4 to 6 min. The measured DBC values were summarized in Table 2. Overall, the DBC of canine MAb A on MabSelect SuRe column increased about 4 fold by increasing protein concentration from 0.8 g/L to 5.4 g/L and by adding 1 M (NH₄)₂SO₄ into the harvest load.

TABLE 2
Effect of MAb Concentration, Flow Rate and (NH₄)₂SO₄ on Dynamic
Binding Capacities of Canine MAb A on MabSelect SuRe Resin.

| Load Conditions | | | |
|---|---|---|---|
| MAb A Conc. (g/L) | (NH₄)₂SO₄ (M) | RT (min) | DBC (5% BT, g/L) |
| 0.8 | 0 | 4 | 10 |
| 1.6 | 0 | 4 | 13.6 |
| 5.4 | 0 | 4 | 16 |
| 5.3 | 1 | 4 | 44 |
| 5.3 | 1 | 6 | 41 |
| 1.7 | 1 | 6 | 38 |

### Example 3

### Effect of Various Kosmotropic Salt on Dynamic Binding Capacity of Canine MAb A on MabSelect SuRe Protein A Resin

Apart from (NH₄)₂SO₄, Na₂SO₄ and NaCitrate were also evaluated in DBC experiments for canine MAb A on the MabSelect SuRe resin. The feed preparation was similar to that described in Example 2, except that the concentrated clarified harvest was supplemented with a concentrated Na₂SO₄ or NaCitrate stock solution to obtain final salt concentration of 0.5 or 0.3 M and protein concentration of 4.8-5.5 g/L. For comparison, a condition at 0.5 M (NH₄)₂SO₄ at similar protein concentration was also conducted in this set of runs. The DBC experiments were performed at flow rate corresponding to 4 to 6 min RT.

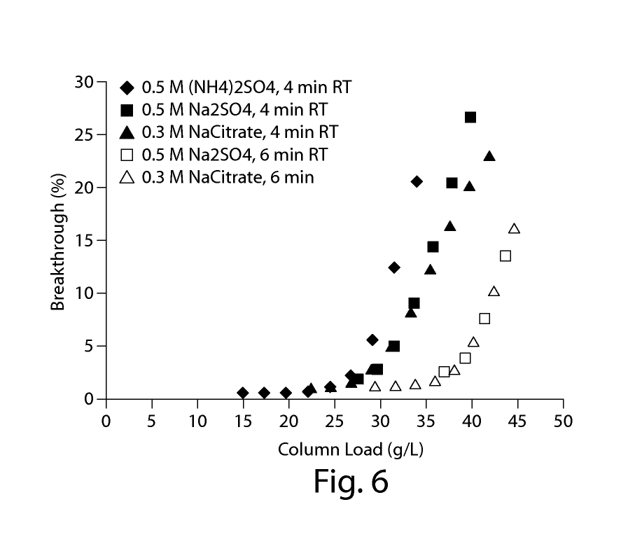

FIG. 6 shows the breakthrough curves for canine MAb A on MabSelect SuRe Protein A resin when the feed contains 0.5 M (NH₄)₂SO₄, 0.5 M Na₂SO₄, or 0.3 M NaCitrate. Consistent with the static binding capacity results, both Na₂SO₄ and NaCitrate give higher DBC than (NH₄)₂SO₄ at the same flow rate and similar salt concentrations. The DBC at 5% breakthrough was 29.1 g/L for 0.5 M (NH₄)₂SO₄, 31.6 g/L for 0.5 M Na₂SO₄ and 31.1 g/L for 0.3 M NaCitrate at 4 min RT flow rate, and 39.2 g/L for 0.5 M Na₂SO₄ and 40.3 g/L for 0.3 M NaCitrate at 6 min RT. Again, it shows that NaCitrate is most effective in enhancing MAb A binding capacity because the higher binding capacity was obtained with the least salt concentration (e.g. 0.3 M). In comparison, a 0.5 M Na₂SO₄ or higher concentration (>0.5 M) of (NH₄)₂SO₄ is needed to achieve similar DBC.

### Example 4

### Effect of (NH₄)₂SO₄ Concentration on MabSelect SuRe Protein A Resin Performance for Canine MAb A

The capture performance of MabSelect SuRe Protein A resin was evaluated at various concentrations of (NH₄)₂SO₄ for canine MAb A. The DBC experiments were assessed at (NH₄)₂SO₄ concentration of 0 to 1 M. In this set of experiments, the equilibration and wash buffer contained the same concentration of (NH₄)₂SO₄ as that in the load sample, which was prepared by pre-concentration of a low titer harvest and supplemented with a stock (NH₄)₂SO₄ solution to get to the targeted salt and protein concentrations (as described in Example 2). The protein concentrations ranged from 4.7 to 5.8 g/L. After equilibration with the respective buffer, the column was loaded with the conditioned feed until breakthrough occurred or slightly before breakthrough. The column was then washed with 6 CV of the equilibration buffer, and then eluted with 5 CV of 20 mM Tris, pH 8.5 solution. The eluate pool was collected based on UV280 from 200 mAU to 200 mAU. The column was then regenerated with 0.15 M phosphoric acid followed by 0.1 N NaOH cleaning before next use. All steps were operated at 4 min RT flow rate. In this case, the eluate pool was collected and analyzed by Poros G assay to determine the protein concentration and by an in-house HCP ELISA assay to quantify the HCP levels. In the case that breakthrough was not occurred, the DBC value should be greater than that determined from the eluate protein concentration.

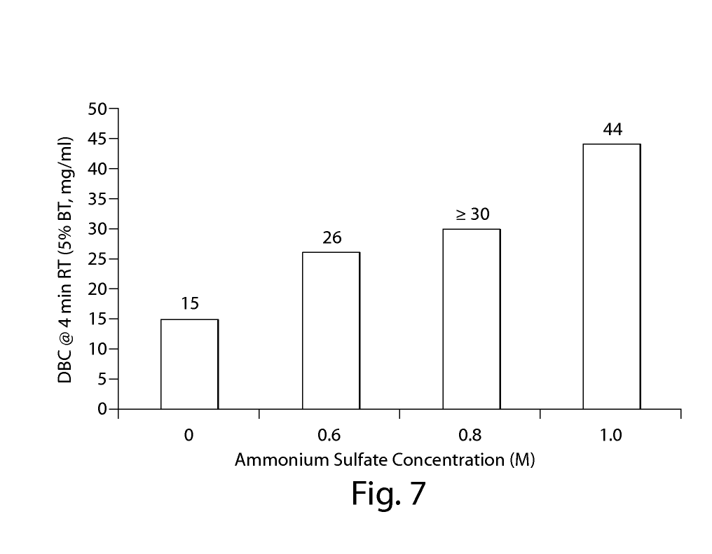

The effect of (NH₄)₂SO₄ concentration on the DBCs of MabSelect SuRe resin was shown in FIG. 7. The differences in the load MAb concentration should have no effect on the DBC, according to results shown in Example 3, thus, the capacity differences observed here were due to the effect of (NH₄)₂SO₄. As expected, increasing (NH₄)₂SO₄ concentration has a large impact on the DBCs for canine MAb A. An approximately 3-fold improvement on the DBC was observed when (NH₄)₂SO₄ concentration increased from 0 to 1 M. Thus, adjusting kosmotropic salt concentration can be used to modulate the binding capacity of a Protein A resin for this weakly associated antibody molecule.

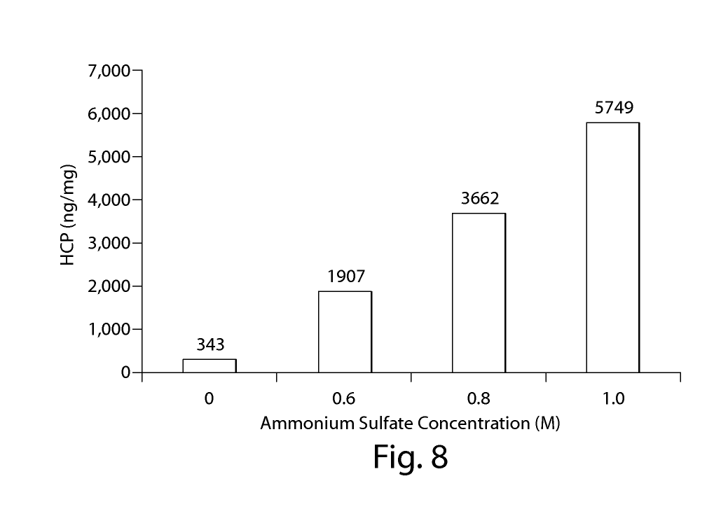

FIG. 8 showed the HCP levels in the eluate pool during MAbSelect SuRe capture purification of the canine MAb A in the presence of various concentrations of (NH₄)₂SO₄. Similar to MAb A, an increased binding of HCP to the resin was also observed as (NH₄)₂SO₄ concentration increased. However, such HCP levels were still within the range typically observed for a MAb on Protein A resin. Selecting an appropriate (NH₄)₂SO₄ concentration is critical to meet both throughput and product quality requirements. Same conclusion can be drawn for other kosmotropic salts given their similar behavior on the binding capacity.

### Example 5

### Canine MAb A Purification by a Two-Column Process Based on (NH₄)₂SO₄-Assisted Protein A Capture

A 50 L canine MAb A bioreactor harvest was clarified by using 0.55 m² of D0HC followed by 0.33 m² of X0HC Pod depth filter and 0.1 m² Sartopore 2 0.45/0.2 um sterile filter cartridge. The clarified harvest (˜1.0 g/L titer) was first concentrated by approximately 11-fold using a 30 kD Biomax membrane cassette. The concentrated harvest was diluted to 3 mg/ml, then supplemented with 0.1% (v/v) Triton X-100. It was then diluted with 40 mM Tris, 2.2 M (NH₄)₂SO₄, pH 7.5 solution to obtain final protein concentration of 2.5 g/L and (NH₄)₂SO₄ concentration of 0.5 M. This material was then 0.22 um filtered to remove haziness.

A 1.0 cm (i.d.)×22 cm MabSelect SuRe column was pre-conditioned with 0.1 N NaOH followed by equilibration with 5 CV of 20 mM Tris, 0.5 M (NH₄)₂SO₄, pH 7.5 buffer. The column was then loaded with the (NH₄)₂SO₄-conditioned harvest (titer 2.5 g/L) to a total loading level of 26 g/L using staged flow rate: 0-20 g/L at 330 cm/hr and 20-26 g/L at 220 cm/hr. The column was then washed with 5 CV of 20 mM Tris, 0.8 M (NH₄)₂SO₄, pH 7.5 buffer followed by 1 CV of 20 mM Tris, 0.5 M (NH₄)₂SO₄, pH 7.5 buffer at 330 cm/hr prior to elution with 5 CV of 20 mM Tris, pH 8.5 buffer. The elution pool was collected based on UV280 from 500 to 500 mAU. After elution, the column was regenerated with 3 CV of 0.15 M phosphoric acid and cleaned with 5 CV of 0.1 M NaOH at 380 cm/hr. The column was re-equilibrated before the next cycle. Five cycles were run to generate enough materials for downstream processing.

The protein A eluates were combined and conditioned to final conductivity of 28 mS/cm and pH 8. The conditioned feed, with total mass of 1.6 g, was then filtered through a 26 cm² X0HC μPod device at ˜100 LMH flow rate. After feed load, the filter was flushed with 52 ml of 20 mM Tris, 0.1 M (NH₄)₂SO₄, pH 8 buffer to recover any bound product.

The filtrate was diluted with 20 mM Tris, pH 8 buffer to achieve conductivity of 6 mS/cm at pH 8 for further polishing through a 5 ml prepacked Capto Q column (GE Healthcare). The column was cleaned with 0.1 N NaOH, equilibrated with 5 CV of 25 min Tris, 27 min NaCl, pH 8 (6 mS/cm) buffer, then loaded with the diluted X0HC filtrate to about 40 g/L loading level at staged flow rate (0-33 g/L at 1.25 ml/min and 33-40 g/L at 0.5 ml/min) The column was washed with 8 CV of equilibration buffer and eluted with 50 mM Tris, 280 min NaCl, pH 7.5 buffer (32.5 mS/cm) at 1.25 ml/min. The elution pool was collected based on UV280 from 200 to 200 mAU. The column was then stripped with 5 CV of 50 mM Tris, 1 M NaCl, pH 7.5 buffer followed by cleaning with 5 CV of 0.5 N NaOH at 2.5 ml/min flow rate.

The eluate or filtrate samples were taken from each step for yield and purity analyses. The protein concentration was measured by UV280 and Poros G assay. The monomer/aggregates levels were determined by SEC, HCP and leached protein A by in-house ELISA assays.

Table 3 summarizes the step yield and impurity level from each step. The step yield for harvest clarification was ˜74%, slightly lower than one would expect. This is due to lack of buffer flush of the filter after loading the harvest sample. The yields for all the other steps were within typical range for the respective operations, and were all above 90%. The MabSelect SuRe column effectively removed the majority of the HCPs, from the initial 200,000 ng/mg in the load to <400 ng/mg in the Protein A eluate, representing a 2.6 log clearance. The X0HC provided additional one log reduction on the HCP level and the Capto Q resin further reduced it to less than 10 ng/mg. The final product has a monomer level over 99% (with aggregates less than 1%) and leached protein A below quantitation limit

TABLE 3
Purification Performances of a Two-
Column Process for Canine MAb A.

| | Yield | HCP | Monomer | Aggregate | Protein A |
|---|---|---|---|---|---|
| Step | (%) | (ng/mg) | (%) | (%) | (ng/mg) |
| Clarification | 74 | ND | NA | NA | NA |
| MabSelect | 100 | 158774-211622 | NA | NA | NA |
| SuRe | | | | | |
| Protein | | | | | |
| A load | | | | | |
| preparation | | | | | |
| (NH₄)₂SO₄- | 90 | 238-391 | 98.7 | 1.01 | 4.59 |
| assisted | | | | | |
| MabSelect | | | | | |
| SuRe | | | | | |
| Protein | | | | | |
| A capture | | | | | |
| X0HC | 90 | 18 | 98.8 | 0.80 | LTQ* |
| filtration | | | | | |
| Capto Q | 90-95 | 6 | 99.1 | 0.86 | LTQ* |
| bind-elute | | | | | |
| polishing | | | | | |

*LTQ denotes less than quantitation limit.

### Example 6

### Canine MAb A Purification by a Three-Column Process Based on (NH₄)₂SO₄-Assisted Protein A Capture

The MabSelect SuRe protein A eluate obtained from the experiments shown in Example 5 was also purified through a 5 mL prepacked Capto Phenyl column which was run in flow-through mode. Specifically, the Protein A eluate was first diluted with a 20 min Tris, pH 7.5 buffer to achieve final conductivity ˜23 mS/cm and MAb concentration ˜10 mg/ml. The Capto Phenyl column was cleaned with 0.1 M NaOH followed by equilibration with 5 CV of 20 mM Tris, 0.1 M (NH₄)₂SO₄, pH 7.5 buffer. The column was then loaded with the diluted feed to 80 g/L loading level at 4 min RT flow rate. After that, the column was washed with 10 CV equilibration buffer at the same flow rate. The flow-through pool was collected during the load when UV280 reached 200 mAU and stopped during the wash when UV280 reading dropped back to 200 mAU.

The Phenyl eluate was then conditioned to pH 8, 6 mS/cm and purified through the Capto Q column as described in Example 5. Again, the eluate samples were taken from each step for yield and purity (HCP and aggregates/monomer) analyses.

Table 4 summarizes the purification performance for this three-column process. In this case, Capto Phenyl column plays the same role in terms of impurity clearance as the X0HC filter shown in Example 5. This resin also provided one log reduction for HCP at high step yield (97%). The final product after the Capto Q polishing step has ˜3 ng/mg HCP and 0.45% aggregates (monomer level 99.5%).

TABLE 4
Purification Performances of a Three-Column Process for Canine
MAb A.

| | Yield | HCP | Monomer | Aggregate |
|---|---|---|---|---|
| Step | (%) | (ng/mg) | (%) | (%) |
| Clarification | 74* | ND | NA | NA |
| MabSelect SuRe | 100 | 158774-211622 | NA | NA |
| Protein A load | | | | |
| preparation | | | | |
| (NH₄)₂SO₄-assisted | 104 | 552 | 99.0 | 0.87 |
| MabSelect SuRe | | | | |
| Protein A capture | | | | |
| Capto Phenyl flow- | 97 | 51 | 99.2 | 0.64 |
| through | | | | |
| Capto Q bind-elute | 93 | 3 | 99.5 | 0.45 |
| polishing | | | | |

### Example 7

### Canine MAb A Purification by an Alternative Two-Column Process Based on Na₂SO₄-Assisted Protein A Capture

A two-column process alternative to that described in Example 5 was used to purify canine MAb A. The major difference for this process was the use of Na₂SO₄ instead of (NH₄)₂SO₄ in the MabSelect SuRe Protein A operation. The pre-concentrated canine MAb A (as described in Example 5) was supplemented with 0.05% Triton X-100 and then 0.5 M Na₂SO₄; the protein concentration was adjusted to 5.8 g/L. The 1.0 cm (i.d.)×22 cm MabSelect SuRe column was pre-conditioned with 0.1 N NaOH followed by equilibration with 5 CV of 20 mM Tris, 0.8 M Na₂SO₄, pH 7.5 buffer. The column was then loaded with the Na₂SO₄-conditioned harvest to a total loading level of ˜44 g/L using staged flow rate: 0-24 g/L at 335 cm/hr and 24-44 g/L at 220 cm/hr. The column was then washed with up to 6 CV of 20 mM Tris, 0.8 M Na₂SO₄, pH 7.5 buffer prior to elution with 5 CV of 20 mM Tris, pH 8.5 buffer. The elution pool was collected based on UV280 from 500 to 500 mAU. The column regeneration and cleaning steps were performed identical to that shown in Example 5.

The Protein A eluates were pooled and adjusted to pH 8 and 29 mS/cm for X0HC filtration step. The actual loading level on the X0HC filter was ˜409 g/m². The X0HC filtrate was then purified through Capto Q column. The operating procedures for both X0HC and Q steps were similar to those shown in Example 5. The samples from each step were analyzed to determine the yield, HCP and monomer/aggregates levels.

Table 5 summarized the performance data for Na₂SO₄-based two-column process. Again, all step recoveries were within expected range. The Na₂SO₄-assisted Protein A step allows high loading level but resulted in higher HCP, as one would have expected. This relatively higher HCP level in the MabSelect SuRe eluate can be effectively reduced by the X0HC and Capto Q polishing steps. The final product contained ˜28 ng/mg HCP and ˜1.5% aggregates. The increased aggregate levels in X0HC filtrate and Capto Q elute were due to sample aging for extended period of time before proper SEC analysis was run. Nevertheless, the product quality is within acceptable range for this molecule.

TABLE 5
Purification Performances of an Alternative Two-Column Process for
Canine MAb A.

| | | | Monomer | Aggregate |
|---|---|---|---|---|
| Step | Yield (%) | HCP (ng/mg) | (%) | (%) |
| Clarification | 74* | ND | NA | NA |
| MabSelect SuRe | 100 | 158774-211622 | NA | NA |
| Protein A load | | | | |
| preparation | | | | |
| Na₂SO₄-assisted | 93-105 | 1862-2531 | 96.5-96.9 | 1.0-1.1 |
| MabSelect SuRe | | | | |
| Protein A capture | | | | |
| X0HC filtration | 94 | 616 | 97.8* | 1.6* |
| Capto Q bind- | 84 | 28 | 98.2* | 1.5* |
| elute polishing | | | | |

*material aged prior to SEC analysis

### Example 8

### Dynamic Binding Capacity of Canine MAb A on ProSep Ultra Plus Protein A Resin

The DBC of canine MAb A on a ProSep Ultra Plus Protein A (PUP) column was measured using a purified canine MAb A feed in the absence of kosmotropic salt, or in the presence of 1M (NH₄)₂SO₄, 0.3M sodium citrate (NaCitrate) or 0.5M Na₂SO₄. In these experiments, the canine MAb A feed concentration was adjusted to 2.6-2.8 g/L. A 1 mL pre-packed PUP protein A column was first equilibrated with 20 mM Tris, pH 7.5 buffer (for the case of no salt addition) or 20 mM Tris, pH 7.5 buffer supplemented with 1M (NH₄)₂SO₄, or 0.3M sodium citrate, or 0.5M Na₂SO₄, respectively, followed by feed loading at a flow rate corresponding to 3 min residence time (RT). The breakthrough curves were monitored at UV280 and the DBC values at 5% BT were determined accordingly. After feed loading, the PUP column was washed with respective equilibration buffer and then eluted with a 20 mM Tris, pH 8.5 buffer. The column was then regenerated with 0.15 M phosphoric acid before next use.

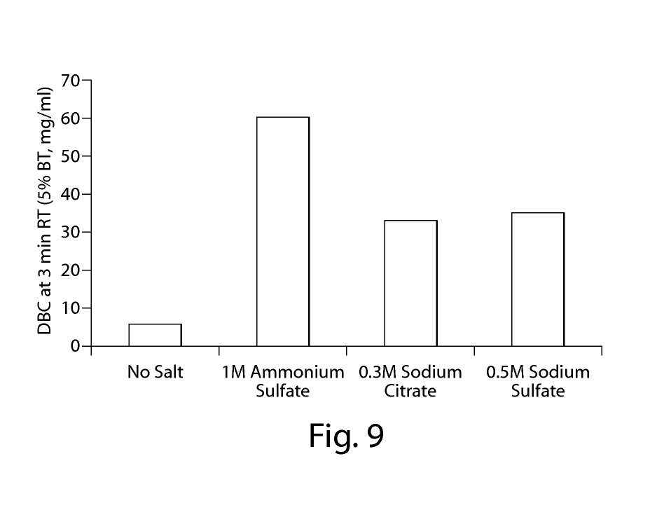

FIG. 9 compares the DBC values for canine MAb A on PUP Protein A column in the absence and presence of various kosmotropic salts at 3 min RT. When there was no salt in the load sample, the canine MAb A capacity was only about 5 g/L resin. In contrast, the DBC increased by over 10-fold when adding 1M (NH₄)₂SO₄ in the load, or increased by over 6-fold when adding 0.3 M Na₂SO₄ or 0.5 M NaCitrate in the load. This data confirm that the increase of canine MAb binding affinity by using kosmotropic salt is independent of the protein A resin used.

1. A method for producing a preparation comprising a non-human antibody, or antigen binding portion thereof, and having a reduced level of at least one impurity, said method comprising:
(a) subjecting a sample comprising the non-human antibody, or antigen binding portion thereof, and at least one impurity to a first kosmotropic salt solution;
(b) contacting the sample subjected to the kosmotropic salt solution to a Protein A affinity chromatography (PA) media; and
(c) obtaining an elution fraction from the Protein A media;
wherein the elution fraction comprises the non-human antibody, or antigen binding portion thereof, and has a reduced level of the at least one impurity.

2. The method of claim 1, wherein the non-human antibody, or antigen binding portion thereof, is
(a) a murine, canine, feline, bovine or equine antibody, or antigen binding portion thereof;
(b) an IgG antibody, or antigen binding portion thereof; and/or
(c) an IgG1 antibody, or antigen binding portion thereof.

3-6. (canceled)

7. The method of claim 1, wherein
(a) the non-human antibody, or antigen binding portion thereof, has a static binding capacity less than about 5 g, about 10 g, about 15 g, about 20 g, or about 25 g of antibody, or antigen binding portion thereof, per one liter of Protein A media;
(b) the static binding capacity of the non-human antibody, or antigen binding portion thereof, increases by at least about 10%, about 25%, about 50%, about 75%, about 100%, about 150%, about 200%, about 300%, or about 400% when the sample is subjected to a kosmotropic solution;
(c) the non-human antibody, or antigen binding portion thereof, has a dynamic binding capacity less than about 5 g, about 10 g, about 15 g, about 20 g, or about 25 g of antibody, or antigen binding portion thereof, per one liter of Protein A media;
(d) the dynamic binding capacity of the non-human antibody, or antigen binding portion thereof, increases by at least about 10%, about 25%, about 50%, about 75%, about 100%, about 150%, about 200%, about 300%, or about 400% when the sample is subjected to a kosmotropic solution;
(e) the binding constant (K) of the non-human antibody, or antigen binding portion thereof, is at least 2, 3, 4, 5, 6, 7, 8, 9 or 10 fold lower than the binding constant (K) for a human antibody; and/or
(f) the binding constant (K) of the non-human antibody, or antigen binding portion thereof, increases by at least about 10%, about 25%, about 50%, about 75%, about 100%, about 150%, about 200%, about 300%, or about 400% when the sample is subjected to a kosmotropic solution.

8-12. (canceled)

13. The method of claim 1, wherein the first kosmotropic salt solution comprises a salt selected from the group consisting of a sulfate salt, a citrate salt, a phosphate salt, ammonium sulfate, sodium sulfate, sodium citrate, potassium sulfate, potassium phosphate, sodium phosphate or a combination thereof.

14. (canceled)

15. The method of claim 1, wherein
(a) the sample is contacted to the Protein A chromatography media in the presence of a load buffer;
(b) the Protein A chromatography media is exposed to an equilibration buffer and/or a wash buffer;
(c) the elution fraction is obtained by contacting the Protein A chromatography media to an elution buffer;
(d) at least one of the load buffer, equilibration buffer and/or wash buffer comprise a second kosmotropic salt solution;
(e) each of the load buffer, equilibration buffer and wash buffer comprise the second kosmotropic salt solution;
(f) the load buffer, equilibration buffer and wash buffer comprise the same or substantially the same second kosmotropic salt solution;
(g) the second kosmotropic salt solution of (d)-(f) comprises a salt selected from the group consisting of a sulfate salt, a citrate salt, a phosphate salt, ammonium sulfate, sodium sulfate, sodium citrate, potassium sulfate, potassium phosphate, sodium phosphate or a combination thereof;
(h) the first kosmotrophic salt solution and the second kosmotropic salt solution of (d)-(g) are the same or substantially the same;
(i) the first kosmotrophic salt solution and/or the second kosmotropic salt solution of (d)-(h) comprise ammonium sulfate, sodium sulfate and/or sodium citrate; and/or
(j) the first kosmotrophic salt solution and/or the second kosmotropic salt solution of (d)-(i) has a concentration of between about 100 mM and 1500 mM.

16-27. (canceled)

28. The method of claim 1, wherein
(a) the equilibration buffer, load buffer and/or the wash buffer have a pH between about 4.0 and 8.5 or between about 5.0 and 7.0;
(b) the equilibration buffer, load buffer and the wash buffer are the same;
(c) the equilibration buffer, load buffer and the wash buffer are substantially the same; and/or
(d) the salt concentration and/or the pH of the equilibration buffer, load buffer and/or wash buffer are within about 50%, 40%, 30%, 20%, 15%, 10% or 5% of the salt concentration and/or pH of each other.

29-31. (canceled)

32. The method of claim 1, wherein the sample has a protein concentration greater than about 1 g/L, about 2 g/L, about 3 g/L, about 4 g/L, about 5 g/L, about 6 g/L, about 7 g/L, about 8 g/L, about 9 g/L or about 10 g/L.

33. The method of claim 1,
(a) wherein the elution fraction is substantially free of the at least one impurity;
(b) the at least one impurity is a host cell protein; and/or
(c) the impurity is a process-related impurity, optionally, selected from the group consisting of a host cell protein, a host cell nucleic acid, a media component, and a chromatographic material.

34-36. (canceled)

37. The method of claim 1, wherein the non-human antibody, or antigen binding portion thereof,
(a) is a humanized antibody or antigen-binding portion thereof, a chimeric antibody or antigen-binding portion thereof, or a multivalent antibody;
(b) comprises a heavy chain constant region selected from the group consisting of IgG1, IgG2, IgG3, IgG4, IgM, IgA and IgE constant regions; and/or
(c) is selected from the group consisting of a Fab fragment, a F(ab′)2 fragment, a single chain Fv fragment, an SMIP, an affibody, an avimer, a nanobody, and a single domain antibody.

38-39. (canceled)

40. The method of claim 1, further comprising repeating steps (a)-(c) of claim 1 at least 1, 2, 3, 4, 5, 6, 7, 8, 9, 10, 11, 12, 13, 14, 15 or 20 times using the elution fraction having a reduced level of the at least one impurity.

41. The method of claim 1, wherein
(a) upon contacting the sample subjected to the kosmotropic salt solution to a Protein A media, a substantial portion of the non-human antibody, or antigen binding portion thereof, binds to the Protein A media,
optionally, wherein the substantial portion of the non-human antibody, or antigen binding portion thereof, is at least about 50%, at least about 60%, at least about 70%, at least about 80%, at least about 90%, or at least about 100% of the antibody, or antigen binding portion thereof, in the sample;
(b) upon obtaining an elution fraction from the Protein A media, a substantial portion of the non-human antibody, or antigen binding portion thereof, is released from the Protein A media,
optionally, wherein the substantial portion of the non-human antibody, or antigen binding portion thereof, released from the Protein A media is at least about 50%, at least about 60%, at least about 70%, at least about 80%, at least about 90% or about 100% of the amount of antibody, or antigen binding portion thereof, bound to the Protein A media;
(c) the yield of the non-human antibody, or antigen binding portion thereof, in the elution fraction is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%; and/or
(d) upon contacting the sample subjected to the kosmotropic salt solution to a Protein A media, a substantial portion of the at least one impurity flows through the Protein A media,
optionally, wherein the substantial portion of the at least one impurity that flows through the Protein A media is at least about 50%, at least about 55%, at least about 60%, at least about 65%, at least about 70%, at least about 75%, at least about 80%, at least about 85%, at least about 90%, at least about 95% or about 100% of the at least one impurity in the sample.

42-47. (canceled)

48. The method of claim 1, wherein the Protein A media is selected from the group consisting of MabSelect SuRe™ MabSelect, MabSelect SuRe LX, MabSelect Xtra, rProtein A Sepharose Fast Flow, Poros® MabCapture A, Amsphere™ Protein A JWT203, ProSep HC, ProSep Ultra, and ProSep Ultra Plus.

49. (canceled)

50. The method of claim 1, wherein
(a) about 10 g to about 100 g of the sample is contacted per one liter of Protein A media;
(b) about 10 g to about 100 g of the non-human antibody, or antigen binding portion thereof, is contacted per one liter of HIC media;
(c) the concentration of the at least one impurity in the sample is about 100 ng to about 300 ng/mg antibody;
(d) the level of the at least one impurity is reduced by at least 80%, at least 90%, at least 95%, at least 98%, at least 99%, or at least 99.9% of the at least one impurity in the sample; and/or
(e) the at least one impurity is reduced by at least 0.25, at least 0.5, at least 0.75, at least 1.0, at least 1.25, at least 1.5, at least 2.0, at least 2.5, at least 3.0 or at least 3.5 log reduction fraction.

51-54. (canceled)

55. The method of claim 1,
(a) wherein a precursor sample comprising the non-human antibody, or antigen binding portion thereof, has been subjected to hydrophobic interaction chromatography to generate the sample; and/or
(b) further comprising subjecting the preparation comprising a non-human antibody, or antigen binding portion thereof, and having a reduced level of one impurity to hydrophobic interaction chromatography, and
optionally, wherein the hydrophobic interaction media is selected from the group consisting of CaptoPhenyl, Phenyl Sepharose™ 6 Fast Flow with low or high substitution, Phenyl Sepharose™ High Performance, Octyl Sepharose™ High Performance, Fractogel™ EMD Propyl, Fractogel™ EMD Phenyl, Macro-Prep™ Methyl, Macro-Prep™ t-Butyl, WP HI-Propyl (C3)™, Toyopearl™ ether, Toyopearl™ phenyl, Toyopearl™ butyl, ToyoScreen PPG, ToyoScreen Phenyl, ToyoScreen Butyl, ToyoScreen Hexyl, HiScreen Butyl FF, HiScreen Octyl FF, and Tosoh Hexyl.

56-57. (canceled)

58. The method of claim 1,
(a) wherein a precursor sample comprising the non-human antibody, or antigen binding portion thereof, has been subjected to ion exchange chromatography to generate the sample; and/or
(b) further comprising subjecting the preparation comprising a non-human antibody, or antigen binding portion thereof, and having a reduced level of one impurity to ion exchange chromatography,
optionally, wherein ion exchange chromatography is performed using ion exchange chromatography media selected from the group consisting of a cation exchange media and an anion exchange media,
optionally, wherein the ion exchange media is an anion exchange media comprising diethylaminoethyl (DEAE), quaternary aminoethyl (QAE) or quaternary amine (Q) group ligands, and
optionally, wherein the ion exchange media is a cation exchange media comprising carboxymethyl (CM), sulfoethyl (SE), sulfopropyl (SP), phosphate (P) or sulfonate (S) ligands.

59-62. (canceled)

63. The method of claim 1,
(a) wherein a precursor sample comprising the non-human antibody, or antigen binding portion thereof, has been subjected to mixed mode chromatography to generate the sample; and/or
(b) further comprising subjecting the preparation comprising a non-human antibody, or antigen binding portion thereof, and having a reduced level of one impurity to mixed mode chromatography, and
optionally, wherein the mixed mode chromatography is performed using CaptoAdhere resin.

64-65. (canceled)

66. The method of claim 1,
(a) wherein a precursor sample comprising the non-human antibody, or antigen binding portion thereof, has been subjected to a filtration step to generate the sample; and/or
(b) further comprising subjecting the preparation comprising the non-human antibody, or antigen binding portion thereof, and having a reduced level of one impurity to a filtration step,
optionally, wherein the filtration step is selected from the group consisting of a depth filtration step, a nanofiltration step, an ultrafiltration step, and an absolute filtration step, or a combination thereof.

67-68. (canceled)

69. A pharmaceutical composition comprising the preparation produced by the method of claim 1 and a pharmaceutically acceptable carrier.

70. A pharmaceutical composition comprising a non-human antibody, or antigen binding portion thereof, and a reduced level of at least one impurity.

71. The pharmaceutical composition of claim 70, wherein the non-human antibody, or antigen binding portion thereof,
(a) is selected from the group consisting of a murine, canine, feline, bovine or equine antibody, or antigen binding portion thereof; and/or
(b) is an IgG antibody, or antigen binding portion thereof, optionally IgG1.

72-74. (canceled)

75. The pharmaceutical composition of claim 70,
(a) wherein the impurity is a host cell protein;
(b) wherein the composition comprises less than about 10%, 9%, 8%, 7%, 6%, 5%, 4%, 3%, 2%, 1.5%, 1.4%, 1.3%, 1.2%, 1.1%, 1%, 0.5%, or less total impurities; and/or
(c) comprising a canine IgG antibody, or antigen binding portion thereof, and having less than about 10%, 9%, 8%, 7%, 6%, 5%, 4%, 3%, 2%, 1.5%, 1.4%, 1.3%, 1.2%, 1.1%, 1%, 0.5%, of host cell protein.

76-77. (canceled)
